(19) United States
(12) Patent Application Publication
(10) Pub. No.: US 2024/0107956 A1
(43) Pub. Date: Apr. 4, 2024

(54) METHOD FOR EXCLUDING THE AGGRESSIVE INCOMPATIBILITY TRAIT FROM STRAINS OF AGARICUS BISPORUS, AND RELATED STRAINS AND LINES

(71) Applicant: Sylvan Inc., Kittanning, PA (US)

(72) Inventors: Michael KESSLER, Kittanning, PA (US); Michelle SCHULTZ, New Bethlehem, PA (US); Mark LOFTUS, Oakmont, PA (US); Aniça AMINI, Saint Pierre Des Corps (FR); Sylvie DELBECQUE, Langeais (FR); Wes SCHULTZ, Worthington, PA (US); Richard KERRIGAN, West Point, CA (US); Mark WACH, Allison Park, PA (US)

(21) Appl. No.: 18/340,136

(22) Filed: Jun. 23, 2023

Related U.S. Application Data
(60) Provisional application No. 63/355,283, filed on Jun. 24, 2022.

(51) Int. Cl. A01G 18/00; A01H 15/00

(52) U.S. Cl. CPC A01G 18/00; A01H 15/00

(57) ABSTRACT

A method for excluding an aggressive incompatibility (AI) trait from Agaricus bisporus mushroom strains, wherein the method includes mating a culture of a hybrid mushroom line designated B18287-s82, a representative culture of the line having been deposited under NRRL Accession No. 68168, with a culture of the white mushroom line designated WBL-s290, to obtain an F1 hybrid mushroom strain designated J19109, a representative culture of the F1 strain having been deposited under NRRL Accession No. 68163. Upon fruiting a culture of the new F1 strain designated J19109 to obtain homokaryotic spores therefrom, one obtains cultures of homokaryotic lines from the homokaryotic spores from F1 strain J19109 and selects a culture of a homokaryotic line from the F1 strain J19109. The culture of a homokaryotic line from F1 strain J19109 is mated with a culture of the mushroom line designated J11500-s80, to obtain an F2 hybrid mushroom strain. The culture of the F2 hybrid mushroom strain is tested to determine the presence or absence of the AI trait, wherein, in the absence of the AI trait, the AI trait has been excluded from the F2 hybrid mushroom strain.

### TECHNICAL FIELD

This invention relates generally to a method for excluding an aggressive incompatibility trait (also referred to as the AI trait) from strains of Agaricus bisporus. Various new strains and lines related to the method are also disclosed.

### BACKGROUND OF THE INVENTION

This invention follows the release of an earlier white hybrid mushroom strain developed by the Sylvan America, Inc. designated as J15987 (NRRL Accession Number 67646) as disclosed in U.S. Pat. No. 10,440,930, the disclosure of which is incorporated by reference herein. The J15987 strain had higher yields and produced mushrooms that were of higher quality than the currently dominant white cultivars, meaning that these mushrooms had thicker cap flesh, and had less red coloration as compared to the standard commercial white mushrooms obtained from the commercially-acceptable strain designated A-15. In early tests of the J15987 strain, customers were very happy with the strong combination of yield and quality, which made these mushrooms and this strain originally very commercially acceptable. However, a problem developed. The J15987 strain was found to include an unprecedented phenotype or trait that has been designated as “Aggressive Incompatibility,” which led to problems at the level of the mushroom farm when those farms were also using the A-15 strain or similar strains. Thus, a need exists for a method that will exclude the phenotype or trait of “Aggressive Incompatibility” (as defined below) from white mushroom strains similar to J15987, and to develop these similar white mushroom strains that retain or otherwise provide the good traits of strain J15987 (such as high yield and/or high quality) whilst excluding the Aggressive Incompatibility (AI) trait.

The edible mushroom, Agaricus bisporus (Lange) Imbach var. bisporus, a basidiomycete fungus, is grown worldwide. In Europe and the Americas, it is the most widely cultivated mushroom species. The value of the annual crop in the United States was worth around $1 billion in 2020-21 (National Agricultural Statistics Service/USDA data).

Cultures of Agaricus are prepared, maintained, propagated, and stored on sterile media in a similar fashion to other microorganisms. Sterile tools and aseptic techniques are used within sterile zones to manipulate pure cultures for various purposes, including spore germination, making new hybrids and making inoculum. Commercial media is “spawn” which can be of a number of different recipes, most commonly sterile millet or rye. Production of spawn is typically on a large scale. For example, one liter of pure Agaricus inoculum can be used on 14,0000 liters of sterilized spawn. The end user, the mushroom farm, receives pure spawn cultures within sterile packaging.

Mushrooms are cultivated commercially within purpose-built structures on mushroom farms. While there are many variations on methods, the following description is typical. Compost is prepared from waste ligno-cellulosic material such as wheat straw, augmented with nitrogenous material, is finished and moved to be pasteurized under specific conditions (aerobic, temperature between 40 and 50° C.), thereby creating a substrate that favors Agaricus colonization. Mushroom spawn is added and colonizes the substrate over a period of 11 to 17 days at controlled temperature. Once colonization, or ‘spawn run’ is complete, a non-nutritive layer of saturated peat (pH adjusted to between 8 and 8.5 with sugar beet lime) is applied on the surface of the compost to a later of approximately 5 cm. Additionally, casing spawn can be added to the peat or casing layer to accelerate mycelial growth. Once colonization has occurred, the environmental conditions are adjusted, in a process termed ‘flushing’, wherein the air temperature drops to approximately 18-19° C., and the CO₂ falls to around 1300 ppm. On day 13 to 18 after the casing soil was applied, mushrooms will appear in the mushroom house. Mushrooms are harvested in a 3-to-4-day period. Additional flushes or breaks of production are produced and harvested before the compost is removed and replaced on the mushroom farm.

Within the United States, 72% of the total harvest was white Agaricus in 2020-21, with the balance being brown Agaricus varieties. The total value of the US mushroom crop in the same period was over $1 billion. In other territories, there is a stronger preference for white mushrooms. Market requirements in the USA and elsewhere for white mushrooms are narrow and precise for phenotypic traits such as size, shape, whiteness, firmness, and related traits such as extended shelf life. Consequently, new white Agaricus strains should comply with a specific set of market requirements. Strains can be separated on the basis of some traits, for example cap shape (e.g. cap roundness, flesh thickness, stem thickness), color (whiteness), density/firmness and piece weight. Some examples of commercial targets for new white mushroom strains are increased crop yield, altered yield distribution across the flushes of production, disease resistance, insect resistance, improved shelf life, reduced bruising, ease of management, suitability for mechanical harvesting, differential response to stressors (for example temperature or substrate change), seasonal influences, farm practices, and strain incompatibility. Strains can also be differentiated and separated from one another using DNA marker techniques, such as Single Nucleotide Polymorphisms (SNPs). Strains may have different ancestry, which will directly reflect the genotype and phenotype.

Circa 1980, the first two true hybrid strains of A. bisporus were developed by a laboratory in Horst, The Netherlands. These two “Horst” strains, called U1 and U3, are closely related hybrids developed from matings of two existing groups of white strains, the pre-hybrid smooth whites (PHSW) and pre-hybrid off whites (PHOW), as described in M. Imbernon et al., Mycologia, 88 (5), 749-761 (1996), incorporated herein by reference. Over time, hybrid strain U1 became the dominant white genotype in the marketplace.

The parental homokaryons of strain U1 were recovered by deheterokaryotizing the culture. The PHSW parent is H39 and the PHOW parent is H97. H97 was deposited in the Fungal Genetics Stock Center, of Kansas USA by A. Sonnenberg under the Accession Number 10389, and also in the American Type Culture Collection as Accession Number MYA-4626. The H97 genome was sequenced and placed in the public domain by the Joint Genome Initiative (California, USA), and this genome has proven very valuable as a reference for single nucleotide polymorphism (SNP) detection (see below).

Strain U1 is the progenitor of all the white strains currently cultivated in North America and Europe. Many mushroom strains such as A-15 and S-130 meet the criteria for Essentially Derived Varieties (EDVs) of U1, having been developed from spores or tissue from the original (i.e., initial) strain. A group of strains developed either by somatic selection or by spore culture, or related methods of ‘essential derivation’ as discussed below, from a single progenitor, is called a derived lineage group. Except for relatively minor genetic differences, all white strains developed from within the U1 derived lineage group share a single composite N+N heterokaryotic genotype, with the original U1 strain. With the notable exception of China, modern white mushroom cultivation is a monoculture.

Agaricus bisporus has a reproductive syndrome known as amphithallism, in which two distinct life cycles operate concurrently. As in other fungi, the reproductive propagule is a spore. Agaricus produces spores meiotically, on a meiosporangium known as a basidium. In a first life cycle, A. bisporus spores each receive a single haploid post-meiotic nucleus; these spores are competent to mate but not competent to produce mushrooms. These haploid spores germinate to produce homokaryotic offspring or lines which can mate with other compatible homokaryons to produce novel hybrid heterokaryons that are competent to produce mushrooms. Heterokaryons generally exhibit much less ability to mate than do homokaryons. This life cycle is called heteromixis, analogous to outbreeding. This life cycle operates, but typically does not predominate, in strains of Agaricus bisporus var. bisporus.

A second, uniparental life cycle called intramixis, analogous to a form of inbreeding, predominates in most strains of Agaricus bisporus var. bisporus. Most spores receive two post-meiotic nuclei, and most such pairs of nuclei consist of Non-Sister Nuclear Pairs (NSNPs) which have a heteroallelic genotype at most or all centromeric-linked loci including the MAT locus. That MAT genotype determines the consequently heterokaryotic phenotype of these offspring, which are reproductively competent and can produce a crop of mushrooms. Unusually among eukaryotes, relatively little chromosomal crossing-over is observed to have occurred in postmeiotic offspring of A. bisporus; empirically, very little heteroallelism (analogous to heterozygosity) is lost among heterokaryotic intramictic offspring of a heterokaryotic strain.

In both life cycles, a spore is a cell that is a part of the mushroom; that cell is a propagule always having, as its single parent, the mushroom and the culture that produced that mushroom.

With respect to spores, living spores are heterokaryons or homokaryons in a dormant state. Spores are one part of the mushroom organism culture; they incorporate only the genetic material of the single culture that produces them (often called the ‘parent’ for convenience). Other parts of the culture include caps, stems, gills, cells (defined as hyphal compartments incorporating nuclei, mitochondria, cytoplasm, protoplasts, RNA, DNA, proteins, cell membranes, and cell walls including crosswalls), hyphae, and mycelium. Spores may be aseptically collected on sterile material, suspended in sterile water at various dilutions, and plated onto sterile agar growth media in order to produce germinated spores and the cultures incorporated within the spores. A preferred technique is to have within the enclosed petri plate a living Agaricus culture which may stimulate spore germination via the diffusion of a volatile pheromone. Germinated spores may be isolated under a microscope using sterile microtools such as steel needles, onto fresh nutrient agar plates. Using this method, heterokaryotic and homokaryotic offspring of a strain comprising the spores and the cultures incorporated within the spores of said strain may be obtained.

Development of novel hybrid varieties via heteromixis comprises the controlled physical association and mating of two compatible cultures to obtain a novel heterokaryon culture. Homokaryons (i.e., ‘lines’) are the preferred starting cultures for making matings as they have maximal ability to anastomose and achieve plasmogamy with other cultures. Heterokaryons may also be placed in physical contact but with unreasonably low probabilities of a mating resulting in successful formation of a novel heterokaryon. Compatibility is determined by the genotype at the MAT locus; two homokaryons with the same MAT allele cannot establish a heterokaryon after anastomosis, thus homokaryon compatibility represents genetic dissimilarity. In a defined mating program, homokaryotic lines are obtained and are associated in predetermined pairwise combinations. In one method, homokaryon pairs may be placed in close proximity on the surface of a nutrient agar medium in a petri dish and allowed to grow together (in a physical association), at which point anastomoses between the two cultures occur. A successful outcome is a mating that provides a heterokaryon. The novel hybrid heterokaryon may be obtained by transferring mycelium from the fusion zone of the dish.

There is a clear need for commercially acceptable white Agaricus bisporus strains with different genotypes, relative to the U1 derived lineage group, for two reasons. First, strains that are vegetatively incompatible with strains of the U1 derived lineage group are known to retard the spread of virus disease between the strains, and such diseases are known to be a major cause of crop losses in the commercial mushroom industry. Second, it is well known that when an agricultural industry relies extensively on a crop having a single genetic lineage (such as with white A. bisporus in most of the world), there is an increased risk of catastrophic crop failure on a facility-wide or industry-wide scale. Thus, from a food security and risk management perspective, it is highly desirable to simultaneously provide both genetic diversification and commercially acceptable performance and crop characteristics.

As noted above, the present invention was a direct consequence of the commercial release of hybrid strain J15987 (NRRL Accession Number 67646), which is a white hybrid strain (U.S. Pat. No. 10,440,930). Pre-commercial testing demonstrated that compared with other white cultivars such as from strain A-15, strain J15987 had thicker cap flesh and thicker stems. Additionally, color analysis using a Minolta Chroma Meter on J15987 demonstrated less redness on the mushroom caps post-harvest. The commercial potential was considered important, and the strain was grown in the United States directly after the Patent was filed. It successfully met all the original crop production criteria. However, a significant problem emerged. It was observed that in some mushroom growing ‘houses’ (or rooms), particularly where mushrooms from A-15 strains were produced, there were large areas of compost that were devoid of mushroom growth, both within the compost and the casing layer. It became clear that this observation was abnormal and unusual.

The culture was returned to a test facility, where a suspected antagonistic interaction between strain J15987 and other white cultivars was investigated in greater detail. The genetics of strain-to-strain interactions are governed by the self/non-self-recognition system, which is also known as vegetative incompatibility, or alternatively as heterokaryon incompatibility. It was discovered that strain J15987 had a very aggressive, unusual antagonistic reaction when in contact with other, U1-family essentially derived varieties (EDVs) such as A-15. When spawn was mixed in proportions as low as 99% A-15 to 1% J15987, Agaricus mycelium rapidly died and that the A-15 genotype was eventually displaced by J15987. This interaction has been termed “Aggressive Incompatibility,” and having abbreviated the interaction as “A”, the phenotype has been defined as the “AI” trait. To be clear, unrelated strains of basidiomycetes including Agaricus bisporus normally exhibit an incompatibility reaction, which has been exploited for virus control, but the AI trait is a far more aggressive, extreme and problematic manifestation of the more familiar phenomenon. It is speculated that this trait is under genetic control, as is generally understood to be the case for vegetative incompatibility in Basidiomycetes.

Conventional mixing equipment on mushroom farms tends to be operated such that some degree of mixing between inoculated substrates of different strains can occur. Such events are typically small and have modest negative impacts. Because these areas are small, they make a minimal impact on total yield, and have traditionally been tolerated. However, larger and more extreme AI interaction impacts are too great to be tolerated commercially, and customer assessments were that strain J15987 could not be handled profitably in farm operations where strain A-15 was also under cultivation. Thus, the need exists for a commercially desirable strain or strains having the positive attributes of strain J15987 while also lacking the AI trait.

### SUMMARY OF THE INVENTION

In order to understand and overcome (exclude) the underlying genetic basis responsible for the manifestation of the AI trait in strain J15987, a series of matings were developed and the resulting hybrids analyzed. The mating scheme is described in Scheme I below. Essentially, a wild variation line (i.e., B18287-s82, NRRL Accession No. 68168) was combined with one of the homokaryotic parents of strain J15987 (i.e., WBL-s290, NRRL Accession No. 68167) to generate a new F1 hybrid strain (i.e., J19109, NRRL Accession No. 68163). Then, a homokaryon (e.g., line J19109-s40, NRRL Accession No. 68165) from that hybrid strain was mated with the other homokaryotic parent of strain J15987 (i.e., J11500-s80, NRRL Accession No. 68164) to provide a new F2 hybrid strain (e.g., J20176, NRRL Accession No. 68166). Confrontations with the F2 hybrid strain were made to demonstrate the presence or absence of the AI trait. Based upon these confrontations, it became clear that the mating scheme was successful, and that the majority of new crosses were free of the AI trait. The pattern of inheritance permitted further development of a model for the genetic control/exclusion of the AI trait.

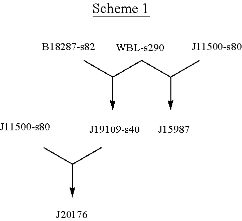

It was first developed that a homokaryon (line B18287-s82) obtained from a hybrid strain developed from mating two homokaryons, namely, W01-s1 and So76-s12b, was necessary. This homokaryotic line, B18287-s82 was developed from parental homokaryons obtained a wild strain W01, which was collected by R. Kerrigan in North America in the 1990s, and a Pre-Hybrid Off White strain So76. DNA testing showed that WO1 was a true wild strain rather than an escaped commercial strain. Additionally, W01 had very similar growing requirements to the current white and brown strains.

Accordingly, one aspect of the present invention is directed generally to a new and distinct homokaryotic line of Agaricus bisporus designated B18287-s82, and processes for using the line designated B18287-s82. In at least one embodiment, a culture including at least one set of chromosomes of the Agaricus bisporus line B18278-s82 is provided, wherein the at least one set of chromosomes comprise all of the alleles of the line B18278-s82 at the sequence-characterized marker loci listed in Table I below. A deposit of a culture of the Agaricus bisporus line B18287-s82, as disclosed herein, has been made with and accepted by the Agricultural Research Services Culture Collection (NRRL), 1815 North University Street, Peoria, Illinois 61604 USA. The date of deposit was Jun. 10, 2022. The culture deposited was taken from the same culture maintained by Sylvan, Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession No. is 68168. The deposit will be maintained in the depository for a period of 30 years, or 5 years after the last request, or for the effective life of the patent, whichever is longer, and will be replaced as necessary during that period. The culture will be irrevocably and without restriction or condition released to the public upon the filing of the patent application or upon the issuance of a patent, whichever is required by the applicable patent laws.

In one embodiment, an Essentially Derived Variety (EDV) of the line B18287-s82 may be provided. Such an EDV may include a culture derived from an initial culture, wherein the initial culture is a culture of line B18287-s82, such that at least 75% of its genome or genotype is present in the genome or genotype of the initial culture of line B18287-s82. In other embodiments, at least 90%, or 95% or 98% or 99%, of its genome or genotype is present in the genome or genotype of the initial culture of line B18287-s82. In other embodiments, such an EDV may include a culture derived directly or solely from the initial culture such that all of its genome or genotype is present in the genome or genotype of the initial culture of line B18287-s82.

The B18287-s82 homokaryon was mated with WBL-s290. It is noted that this line is one of the parental homokaryons presently found in strain J15987 (the other is J11500-s80, which was also used in this invention). The resulting F1 heterokaryon strain was named J19109.

Thus, another aspect of the present invention is directed generally to a new and distinct homokaryotic line of Agaricus bisporus designated WBL-s290, and processes for using the line designated WBL-s290. In at least one embodiment, a culture including at least one set of chromosomes of the Agaricus bisporus line WBL-s290 is provided, wherein the at least one set of chromosomes comprise all of the alleles of the line WBL-s290 at the sequence-characterized marker loci listed in Table I below. A deposit of a culture of the Agaricus bisporus line WBL-s290, as disclosed herein, has been made with and accepted by the Agricultural Research Services Culture Collection (NRRL) 1815 North University Street, Peoria, Illinois 61604 USA. The date of deposit was Jun. 10, 2022. The culture deposited was taken from the same culture maintained by Sylvan, Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession No. is 68167. The deposit will be maintained in the depository for a period of 30 years, or 5 years after the last request, or for the effective life of the patent, whichever is longer, and will be replaced as necessary during that period. The culture will be irrevocably and without restriction or condition released to the public upon the filing of the patent application or upon the issuance of a patent, whichever is required by the applicable patent laws.

In one embodiment, an Essentially Derived Variety (EDV) of the line WBL-s290 may be provided. Such an EDV may include a culture derived from an initial culture, wherein the initial culture is a culture of line WBL-s290, such that at least 75% of its genome or genotype is present in the genome or genotype of the initial culture of line WBL-s290. In other embodiments, at least 90%, or 95% or 98% or 99%, of its genome or genotype is present in the genome or genotype of the initial culture of line WBL-s290. In other embodiments, such an EDV may include a culture derived directly or solely from the initial culture such that all of its genome or genotype is present in the genome or genotype of the initial culture of line WBL-s290.

Another aspect of the present invention is generally directed to a new and distinct Agaricus bisporus mushroom strain culture designated J19109; a hybrid strain obtained via the directed mating the two homokaryotic cultures B18287-s82 and WBL-s290. A deposit of a culture of strain J19109 has been made with and accepted by the Agricultural Research Services Culture Collection (NRRL), 1815 North University Street, Peoria, Illinois 61604 USA. The date of deposit was Jun. 10, 2022. The culture deposited was taken from the same culture maintained by Sylvan Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession Number is 68163. The deposit will be maintained in the depository for a period of 30 years, or 5 years after the last request, or for the effective life of the patent, whichever is longer, and will be replaced as necessary during that period. The cultures will be irrevocably and without restriction or condition released to the public upon the filing the priority application or upon the issuance of a patent on this strain according to the patent laws.

In one embodiment, an Essentially Derived Variety (EDV) of the strain J19109 may be provided. Such an EDV may include a culture of Agaricus bisporus derived from an initial culture, wherein said initial culture is a culture of strain J19109, such that at least 75% of its genome or genotype is present in the genome or genotype of the initial culture of strain J19109. Alternatively, other EDVs may include the culture that is derived directly from the initial culture of strain J19109 such that all of its genome or genotype is present in the genome or genotype of the initial culture of strain J19109.

To reiterate, a culture of a hybrid mushroom line designated B18287-s82, a representative culture of the line having been deposited under NRRL Accession No. 68168, was mated with a culture of the white mushroom line designated WBL-s290, a representative culture of the line having been deposited under NRRL Accession No. 68167, to obtain an F1 hybrid mushroom strain designated J19109, a representative culture of which has been deposited under NRRL Accession No. 68163. The culture of the F1 strain designated J19109 was then fruited to obtain homokaryotic spores therefrom, from which homokaryotic lines were obtained. A culture of a homokaryotic line from the F1 strain J19109 was then selected. Notably, the homokaryotic lines from the F1 strain J19109 lack the alleles at the centromere-linked loci of line WBL-s290 on chromosomes 4, 7 and 9.

Upon selection of a culture of a homokaryotic line from the F1 strain J19109, that culture was mated with a culture of the mushroom line designated J11500-s80, which is noted to be the other parent homokaryon presently found in strain J15987. A resulting F2 hybrid mushroom strain was obtained from that mating, and a culture from the resultant F2 hybrid mushroom strain was tested to determine the presence or absence of the AI trait, wherein, in the absence of the AI trait, the AI trait has been excluded from the F2 hybrid mushroom strain.

Thus, another aspect of the present invention is directed generally to a new and distinct homokaryotic line of Agaricus bisporus designated J11500-s80, and processes for using the line designated J11500-s80. In at least one embodiment, a culture including at least one set of chromosomes of the Agaricus bisporus line J11500-s80 is provided, wherein the at least one set of chromosomes comprise all of the alleles of the line J11500-s80 at the sequence-characterized marker loci listed in Table I below. A deposit of a culture of the Agaricus bisporus line J11500-s80, as disclosed herein, has been made with and accepted by the Agricultural Research Services Culture Collection (NRRL) 1815 North University Street, Peoria, Illinois 61604 USA. The date of deposit was Jun. 10, 2022. The culture deposited was taken from the same culture maintained by Sylvan, Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession No. is 68164. The deposit will be maintained in the depository for a period of 30 years, or 5 years after the last request, or for the effective life of the patent, whichever is longer, and will be replaced as necessary during that period. The culture will be irrevocably and without restriction or condition released to the public upon the filing of the patent application or upon the issuance of a patent, whichever is required by the applicable patent laws.

In one embodiment, an Essentially Derived Variety (EDV) of the line J11500-s80 may be provided. Such an EDV may include a culture derived from an initial culture, wherein the initial culture is a culture of line J11500-s80, such that at least 75% of its genome or genotype is present in the genome or genotype of the initial culture of line J11500-s80. In other embodiments, at least 90%, or 95% or 98% or 99%, of its genome or genotype is present in the genome or genotype of the initial culture of line J11500-s80. In other embodiments, such an EDV may include a culture derived directly or solely from the initial culture such that all of its genome or genotype is present in the genome or genotype of the initial culture of line J11500-s80.

It will further be appreciated that, in one or more embodiments, the F2 hybrid strains that exclude the AI trait, will retain at least two beneficial traits found in strain J15987. Such beneficial traits may be selected from the group consisting of a cap shape as round as the cap shape for strain J15987, stems as thick as the stems for strain J15987, flesh as thick as the flesh for strain J15987, and a redness (a) value on the L-a-b color measurement scale that is within 10%, or 5%, or 2.5%, or 1%, or 0.5%, of the redness (a) value for the strain J15987. It will be appreciated that by the terms “as round as” and “as thick as,” it is meant that the caps, stems and/or flesh of the mushrooms fruited by the F2 hybrid strains, as measured, are within the statistical significance of the roundness or thickness of the cap, stems or flesh of the mushrooms fruited by strain J15987. In alternative embodiments, the roundness of the cap shape of a mushroom of the F2 hybrid strain may be within 20%, or 15%, or 10%, or 5%, of the roundness of the cap shape of the mushroom of strain J15987. In other alternative embodiments, the thickness of the stems of the mushrooms of the F2 hybrid strain may be within 20%, or 15%, or 10%, or 5%, of the thickness of the stem of the mushroom of strain J15987. In still other alternative embodiments, the thickness of the flesh of the mushrooms of the F2 hybrid strain may be within 20/6, or 15%, or 10%, or 5%, of the thickness of the flesh of the mushroom of strain J15987.

In one embodiment, the homokaryotic line selected from F1 strain J19109 may be a homokaryotic line designated J19109-s40. It will be appreciated that mating the line culture designated J19109-s40 with the mushroom line designated J11500-s80, wherein a resultant F2 hybrid strain designated J20176. It has been found that the hybrid strain J20176 is free of the AI trait.

Thus, another aspect of the present invention is directed generally to a new and distinct homokaryotic line of Agaricus bisporus designated J19109-s40, and processes for using the line designated J19109-s40. In at least one embodiment, a culture including at least one set of chromosomes of the Agaricus bisporus line J19109-s40 is provided, wherein the at least one set of chromosomes comprise all of the alleles of the line J19109-s40 at the sequence-characterized marker loci listed in Table I below. A deposit of a culture of the Agaricus bisporus line J19109-s40, as disclosed herein, has been made with and accepted by the Agricultural Research Services Culture Collection (NRRL) 1815 North University Street, Peoria, Illinois 61604 USA. The date of deposit was Jun. 10, 2022. The culture deposited was taken from the same culture maintained by Sylvan, Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession No. is 68165. The deposit will be maintained in the depository for a period of 30 years, or 5 years after the last request, or for the effective life of the patent, whichever is longer, and will be replaced as necessary during that period. The culture will be irrevocably and without restriction or condition released to the public upon the filing of the patent application or upon the issuance of a patent, whichever is required by the applicable patent laws.

In one embodiment, an Essentially Derived Variety (EDV) of the line J19109-s40 may be provided. Such an EDV may include a culture derived from an initial culture, wherein the initial culture is a culture of line J19109-s40, such that at least 75% of its genome or genotype is present in the genome or genotype of the initial culture of line J19109-s40. In other embodiments, at least 90%, or 95% or 98% or 99%, of its genome or genotype is present in the genome or genotype of the initial culture of line J19109-s40. In other embodiments, such an EDV may include a culture derived directly or solely from the initial culture such that all of its genome or genotype is present in the genome or genotype of the initial culture of line J19109-s40.

Another aspect of the present invention is generally directed to a new and distinct Agaricus bisporus mushroom strain culture designated J20176; a hybrid strain obtained via the directed mating the two homokaryotic cultures J11500-s80 and J19109-s40. A deposit of a culture of strain J20176 has been made with and accepted by the Agricultural Research Services Culture Collection (NRRL), 1815 North University Street, Peoria, Illinois 61604 USA. The date of deposit was Jun. 10, 2022. The culture deposited was taken from the same culture maintained by Sylvan Inc., Kittanning, Pennsylvania, USA, the assignee of record, since prior to the filing date of this application. All restrictions upon the deposit have been removed, and the deposit is intended to meet all deposit requirements of the U.S. Patent and Trademark Office, including 37 C.F.R. Sec. 1.801-1.809, and all deposit requirements under the Budapest Treaty. The NRRL Accession Number is 68166. The deposit will be maintained in the depository for a period of 30 years, or 5 years after the last request, or for the effective life of the patent, whichever is longer, and will be replaced as necessary during that period. The cultures will be irrevocably and without restriction or condition released to the public upon the filing the priority application or upon the issuance of a patent on this strain according to the patent laws.

In one embodiment, an Essentially Derived Variety (EDV) of the strain J20176 may be provided. Such an EDV may include a culture of Agaricus bisporus derived from an initial culture, wherein said initial culture is a culture of strain J20176, such that at least 75% of its genome or genotype is present in the genome or genotype of the initial culture of strain J20176. Alternatively, other EDVs may include the culture that is derived directly from the initial culture of strain J20176 such that all of its genome or genotype is present in the genome or genotype of the initial culture of strain J20176.

It will further be appreciated that, in one or more embodiments, the F2 hybrid strain designated J20176 excludes the AI trait, and retains at least two beneficial traits found in strain J15987. Such beneficial traits may be selected from the group consisting of a cap shape as round as the cap shape for strain J15987, stems as thick as the stems for strain J15987, flesh as thick as the flesh for strain J15987, and a redness (a) value on the L-a-b color measurement scale that is within 10%, or 5%, or 2.5%, or 1%, or 0.5%, of the redness (a) value for the strain J15987. It will be appreciated that by the terms “as round as” and “as thick as,” it is meant that the caps, stems and/or flesh of the mushrooms fruited by the F2 hybrid strain J20176 are within the statistical significance of the roundness or thickness of the cap, stems or flesh of the mushrooms fruited by strain J15987. In alternative embodiments, the roundness of the cap shape of a mushroom of the F2 hybrid strain J20176 may be within 20%, or 15%, or 10%, or 5%, of the roundness of the cap shape of the mushroom of strain J15987. In other alternative embodiments, the thickness of the stems of the mushrooms of the F2 hybrid strain J20176 may be within 20%, or 15%, or 10%, or 5%, of the thickness of the stem of the mushroom of strain J15987. In still other alternative embodiments, the thickness of the flesh of the mushrooms of the F2 hybrid strain J20176 may be within 20%, or 15%, or 10%, or 5%, of the thickness of the flesh of the mushroom of strain J15987.

It can be seen that the method of the present invention advantageously excludes the AI trait from Agaricus bisporus strains, including at least strain J20176. More particularly, the method is capable of excluding the aggressive incompatibility (AI) trait from Agaricus bisporus mushroom strains descended from both strain J10165 (also called WBL) and strain J11500 (as disclosed in U.S. Pat. No. 9,622,428), wherein it is known that mating a culture of a white mushroom line designated WBL-s290, with a culture of a mushroom line designated J11500-s80, provides a hybrid mushroom strain designated J15987, a representative culture of the strain having been deposited under NRRL Accession No. 67646, that has the AI trait.

With respect to obtaining cultures of homokaryotic lines from the homokaryotic spores from F1 strain J19109, it will be appreciated that, when the J19109 heterokaryon was fruited and F2 spores were collected, an initial total of 52 homokaryons were obtained, and 16 of these are described in several of the Tables below. All 52 homokaryons were mated to the second parent of J15987, J11500-s80, and the resulting strains were extensively screened for commercial potential. Collected data, which is shown below, showed that the beneficial traits of J15987 were maintained and more importantly the majority of the new J19109×J11500-s80 hybrids were free of the AI trait. One hybrid, strain J20176, was selected for controlled testing. The strain performed robustly and is therefore a strong candidate for commercialization.

The entire pool of strain J19109's homokaryotic lines showed similar potential to that of J19109-s40, and all are considered to be valuable breeding lines with the potential to produce high quality mushroom hybrids that lack the AI trait.

One use of the uses of the J19109-derived hybrids is the production of crops of edible mushrooms for sale. Thus, mushrooms obtained from the cultures of any of the lines or strains designated above are a part of this invention. Another use is for the improvement of facility hygiene via strain rotation and a ‘virus-breaking’ effect. A further use is to incorporate the genetic material of strain J19109 into offspring and derived or descended cultures including dormant and germinating spores and protoplasts. Additional uses also exist as noted above.

The unusual biology of fungi, and in particular intramixis in Agaricus bisporus, makes deriving virtual copies of cultures having essentially the same phenotype facile and straightforward. In plant breeding, such copies are referred to as Essentially Derived Varieties, or EDVs. Methods of obtaining cultures which are by definition consequently EDVs of a single initial culture of A. bisporus include somatic selection, tissue culture selection, single spore germination, multiple spore germination, selfing, repeated mating back to the initial culture, mutagenesis, and transformation, to provide some examples. DNA-mediated transformation of A. bisporus has been reported by Velcko, A. J. Jr., Kerrigan, R. W., MacDonald, L. A., Wach, M. P., Schlagnhaufer, C., and Romaine, C. P. 2004, Expression of novel genes in Agaricus bisporus using an Agrobacterium-mediated transformation technique. Mush. Sci. 16: 591-597, and references therein, herein incorporated by reference. Transformation may introduce a single new gene or allele into the genome of an initial culture. Additionally, recent reports on other closely related fungi raise the possibility of gene editing via CRISPR.

EDVs are unambiguously recognizable by their genotype, which will be predominantly or even entirely a subset of the single initial culture. Percentages of the initial genotype that will be present in Agaricus bisporus EDVs range from 100% or virtually 100% in the case of single spore cultures and of somatic selections, to 99.x % in the case of strains modified by DNA-mediated transformation, to 90-99.x % in the case of some single or multiple spore selections or some mutagenesis, to an average of at least 75-85% in the case of sibling-offspring matings (=selfing) and of back-matings to the initial culture. Many methods of genotype determination, including methods described below, and others well known in the art, may be employed to determine the percentage of DNA of an initial culture that is present in another culture, and to make unambiguous determinations of the relationship between two cultures and any methods used to manipulate or exploit an initial culture.

Repeated mating back to the initial culture also produces an EDV of the initial culture. In a hypothetical example, in the first successive iteration of this process a resultant strain of this generation will have on average about 75% of the DNA of the initial strain while about 25% of the DNA will have been contributed by a second strain or line; as this process is repeated the DNA representation of the initial strain will increase, approaching 97% on average after 3 further successive repetitions. Generally, it will be appreciated that any culture having 75-100% genotype identity with an initial culture is indicative of an EDV of an initial culture. It is also established that an EDV of an EDV is also an EDV of an initial strain. Finally, because Agaricus bisporus alternates generations between heterokaryotic strains and homokaryotic lines, the criteria for essential derivation apply equally to cultures of both strains and lines.

Genotypic fingerprints are descriptions of the genotype at defined loci, where the presence of characterized alleles is recorded. Such fingerprints provide powerful and effective techniques for recognizing clones and all types of EDVs of an initial strain, as well as for recognizing ancestry within outbred lineages. Many techniques are available for defining and characterizing loci and alleles in the genotype. The most detailed approach is provided by whole-genome sequencing (WGS), which allows for direct characterization and comparison of DNA sequences across the entire genome. Using this approach to generate robust genotypic fingerprints incorporating large numbers of marker loci, it is possible to establish the nature of the relationship between two strains, including strains related by genealogical descent over several generations. Applicant has tracked genetic markers through four to six generations of its strain development pedigrees. If a sufficient number of distinctive markers are present in an initial strain or line, it will be possible to identify descent from an initial strain or line after several outbred generations without undue experimentation. In a hypothetical example, the mean expectation for genomic representation of an initial haploid line after 4 outbred generations is 3.1% (50%/2⁴) in an F4 hybrid, which corresponds to ca. 1 Mb of the nuclear genomic DNA of A. bisporus. Based on the Applicant's analyses, that amount of DNA from each of two unrelated strains of A. bisporus may typically contain from about 10,000 to about 20,000 Single Nucleotide Polymorphisms (SNPs), any one of which may provide a distinguishing marker linking the F4 hybrid to the initial line. By using multiple independent markers, ancestors of a strain can be identified with a very high probability of success and with reasonable confidence.

One trait of biological and commercial interest is heterokaryon incompatibility. The genetics of these self/non-self-recognition systems are not well elucidated in basidiomycete fungi such as Agaricus however are known in other groups of fungi to involve multiple alleles at multiple independent loci. Note that heterokaryon incompatibility occurs in most fungi, and is quite different from the Aggressive Incompatibility trait reported herein. “Normal” incompatibility is usually a localized area of mycelial interaction, where growth between the two genotypes is attenuated. The function is believed to be to prevent the transfer of viruses between different genotypes of the same species.

It should be noted that there are at least two double stranded RNA viruses that affect mushroom crops, La France disease and Mushroom Virus X. Heterokaryon incompatibility will prevent successful anastomoses and cytoplasmic continuity among physical mixtures of two or more heterokaryons, thereby creating a ‘Virus-break’. On a mushroom farm, virus-breaking is carried out by replacing cropping material (compost, spawn, casing inoculum) incorporating an initial strain with inoculum and cropping material incorporating another different strain that is incompatible with the initial strain. In the most effective implementation of the virus-breaking method, all biological material of the initial strain at a mushroom farm is replaced with biological material of the second, incompatible strain. Strain incompatibility creates an effective if not absolute barrier to movement of virus from biological reservoirs within a facility into new crops. Rotating cultivation usage among mushroom strains of different genotypes may also interrupt infection and infestation cycles of exogenous pests and pathogens.

As noted above, hybrid mushroom strain producers are always looking for hybrid strains that allow growers to produce crops successfully and profitably. In the case of new hybrids derived from J19109, and strains derived or descended thereof, positive attributes documented thus far include an attractive appearance (round caps, thick stem, good bruise resistance, compared with market leader A-15, all of which appeal to customers), and a total yield that can match or exceed that of strains like A-15. In our example, a mushroom grower can obtain a high yielding crop of high-quality mushrooms by using the new hybrids described herein. Additionally, these strains match the high quality of J15987, an earlier strain developed by the Applicant, with the considerable advantage of lacking the Aggressive Incompatibility phenotype.

Strains currently available to the mushroom industry allow growers to produce crops of mushrooms successfully and usually profitably. Several factors exist that influence the degree of success and profitability achieved. For example, a strain must be able to produce at least a comparable crop yield over two to three breaks or flushes, relative to strains currently being marketed and grown commercially. Also, some physical properties of the mushrooms produced, for example cap color category, and general size and dimensions such as cap diameter, allow mushrooms to be marketed in familiar product categories.

J19109 SSI×J11500-s80 hybrids meet those needs of the market and solve the current problem of the unavailability of such a strain to the market. In addition, this group of new strains differ genotypically when compared to other current white cultivars (see Tables I and II below), which addresses and solves potential problems associated with reliance upon a monocultural crop.

One or more aspects of the present invention may be accomplished by a hybrid mushroom culture of Agaricus bisporus developed from this invention. Thus, a product incorporating any of the cultures of the lines or strains above are encompassed by this invention. Such products may include mycelium, spawn, fresh or processed mushrooms, mushroom spores, mushroom spawn, mushroom preparations and extracts and fractions, mushroom pieces, mushroom inoculum, casing inoculum, casing spawn, casing soil, inoculated compost, colonized compost, post-cropped compost and friable particulate matter.

Furthermore, various parts of the cultures of any of the lines or strains above may have value and commercial application. Thus, a part selected from the group consisting of hyphae, mushrooms, spores, cells, nuclei and protoplasts is envisioned. Still further, in one or more embodiments, single cell microorganisms are envisioned. Accordingly, a cell of any of the cultures of the lines and strains above are provided.

It is noted that strain J20176, a representative culture of the strain having been deposited under NRRL Accession No. 68166, has been deposited in order to provide a suitable example of the possible lines meeting the requirements of the present invention. Thus, the strain J20176 may include various parts of the culture, including hyphae, spores, and cells and parts of cells, including, nuclei, mitochondria, cytoplasm, protoplasts, DNA, RNA, proteins, cell membranes and cell walls, said parts being present in both the vegetative mycelium of the culture and in mushrooms produced by the culture. The spores may be dormant or germinated spores and may include heterokaryons and homokaryons incorporated therein.

One or more products incorporating the hybrid mushroom culture of Agaricus bisporus designated as strains descended from J19109 may be produced. Such products include mycelium, spawn, inoculum, casing inoculum, fresh mushrooms, processed mushrooms, mushroom extracts and fractions, mushroom pieces, and colonized substrates selected from grain, compost, and friable particulate matter. It will be appreciated that mushroom pieces refer to stems, pilei, and other larger portions of the mushroom itself.

One or more other aspects of the present invention may be accomplished by an Essentially Derived Variety (EDV) of the hybrid mushroom culture of strain such as J19109. In one or more embodiments, an Agaricus bisporus culture produced by essential derivation has at least one of the essential characteristics of J19109 derived matings. For this invention, we deposited one mating, J19109-s40×J11500-s80, known as J20176 as an example of an elite strain which was a direct result of the invention described herein. J20176 is a strain which lacks the AI trait, and possesses the further characteristics of cap roundness, flesh thickness, yield performance, and yield timing of J15987 relative to U1 EDVs such as A-15.

Other aspects of the present invention may be accomplished by a method for producing a hybrid culture of Agaricus bisporus that includes a step of mating a homokaryon such as J19109-s40 (deposited with NRRL Accession Number 68165) with a second homokaryon. In one embodiment, the second homokaryotic line J11500-s80, a culture of which was deposited at NRRL Accession Number 68164. Such a mating provides the mushroom culture J20176, which demonstrates a normal pattern of antagonism to a panel of commercial cultivars based on U1 EDVs, for example A-15. This antagonism demonstrates the genetic distinctness of strain J20176. In one or more embodiments, the method further includes providing a mushroom culture of the invention consisting of mycelium, spawn, inoculum, casing inoculum, fresh mushrooms, process mushrooms, parts of mushrooms, mushroom extracts and fractions, mushroom pieces, and colonized substrates selected from grain, compost, and friable particulate matter. In other embodiments, the method may include providing the mushroom culture in derived cultures selected from the group consisting of, homokaryons, heterokaryons, aneuploids, somatic subcultures, tissue explant cultures, protoplasts, dormant spores, germinating spores, inbred descendants and outbred descendants, transgenic cultures, gene-edited cultures, and cultures having genomes with a single locus conversion.

Still further aspects of the present invention may be accomplished by a hybrid mushroom culture of Agaricus bisporus having a genotypic fingerprint which has characters at marker loci ITS, p1n150-G3-2, MFPC-1-ELF, AN, AF, and FF. In one or more embodiments, the culture has a genotypic fingerprint having characters at marker loci described in Table II wherein all of the characters of said fingerprint are present in the genotypic fingerprint. To be clear, meiosis and random assortment will ensure that each homokaryotic spore descended from J19109 will have a unique pattern of markers. It will be straightforward to those skilled in the art to deduce that the J19109 homokaryon breeding stocks are descendants, on the basis of the inherited DNA markers.

One or more further aspects of the present invention may be accomplished by a culture, a cell or a culture including the cell, produced by the method(s) above. Thus, one or more embodiments may include a method further including the step of growing the hybrid mushroom culture to produce hybrid mushrooms and parts of mushrooms. Other embodiments may provide for methods wherein the hybrid mushroom culture produced, or the cell, includes a marker profile having characters at marker loci ITS, p1n150-G3-2, MFPC-1-ELF, AN, AF, and FF, wherein all of the characters of said marker profile are also present in the marker profile of strains J20176 and J19109. Still other embodiments may provide for methods wherein the hybrid mushroom culture produced, or the cell, includes a marker profile having characters at marker loci described in Table I wherein all of the characters of said marker profile are also present in the marker profile of strain J20176.

These and other advantages of the present invention over existing prior art relating to Agaricus bisporus mushrooms and cultures, which shall become apparent from the description which follows, are accomplished by the invention as hereinafter described and claimed.

### DETAILED DESCRIPTION OF THE INVENTION

Initially, to provide clear and consistent understanding of the specification and claims, including the scope to be given such terms, the following descriptions are provided.

Allele: One or two or more alternative forms of a gene that arise by mutation and are found at the same place on a chromosome: a heritable unit of the genome at a defined locus, ultimately identified by its DNA sequence (or by other means).

Aggressive Incompatibility: An interaction between two heterokaryons, where the two cultures show strong antagonism towards one another, a reaction that is much more severe than a typical heterokaryon incompatibility reaction. On the level of the mushroom farm, large areas of dead mycelium are seen on both the compost and the casing, and there is a consequent loss of yield of at least 15%, and more commonly, at least 50%. In the lab, the presence of a mere 1% of J15987 (a culture with the Aggressive Incompatibility (AI) trait) can kill a U1 EDV such as A-15. Generally, as noted above, if more than 15% of A-15 is killed by J15987, or other strain, then it is said to have the AI trait. Over time the strain with the AI trait will displace the U1 EDV and will become the only genotype present.

Amphithallism: A reproductive syndrome in which heteromixis and intramixis are both active.

Anastomosis: Fusion of two or more hyphae that achieves cytoplasmic continuity.

Basidiomycete: A monophyletic group of fungi producing meiospores on basidia; a member of a corresponding subdivision of Fungi such as the Basidiomycetales or Basidiomycotina.

Basidium: The meiosporangial cell, in which karyogamy and meiosis occur, and upon which the basidiospores are formed.

Bioefficiency: For mushroom crops, the net fresh weight of the harvested crop divided by the dry weight of the compost substrate at the time of spawning, for any given sampled crop area or compost weight.

Breeding: Development of strains, lines or varieties using methods that emphasize sexual mating.

Cap: Pileus; part of the mushroom, the gill-bearing structure.

Cap Flatness: A measure of the shape or thickness of a mature open mushroom cap.

Cap Roundness: Strictly, a ratio of the maximum distance between the uppermost and lowermost parts of the cap, divided by the maximum distance across the cap, measured on a longitudinally bisected mushroom: typically averaged over many specimens; subjectively, a ‘rounded’ property of the shape of the cap.

Carrier substrate: A medium having both nutritional and physical properties suitable for achieving both growth and dispersal of a culture; examples are substrates that are formulated for mushroom spawn, casing inoculum, and other inoculum.

Casing layer, casing: A layer of non-nutritive material such as peat or soil that is applied to the upper surface of a mass of colonized compost in order to permit development of the mushroom crop.

Casing inoculum (CI): A formulation of inoculum material incorporating a mushroom culture, typically of a defined heterokaryotic strain, suitable for mixing into the casing layer.

Cloning: Somatic propagation without selection.

Combining ability: The capacity of an individual to transmit superior performance to its offspring.

General combining ability is an average performance of an individual in a particular series of matings.

Compatibility: See heterokaryon compatibility, vegetative compatibility, sexual compatibility; incompatibility is the opposite of compatibility.

CRISPR: (Clustered Regularly Interspaced Short Palindromic Repeats) A technique in genetic engineering whereby genomes of living organisms can be modified.

Culture: The tangible living organism; the organism propagated on various growth media and substrates; a portion of, or the entirety of one physical strain, line, homokaryon or heterokaryon; the sum of all of the parts of the culture, including hyphae, mushrooms, spores, cells, nuclei, mitochondria, cytoplasm, protoplasts, DNA, RNA, proteins, cell membranes and cell walls.

Cultivar: Commercially cultivated variety, or strain

Derivation: Development or obtention of a culture solely or predominantly from an initial strain or culture; see EDV. The terms ‘derive’ and ‘derived’ refer to this process or to its outcome.

Derived lineage group: The set of EDVs derived from a single initial strain, and including the initial strain.

Descent: Genealogical descent over a limited number (e.g., 10 or fewer) of generations.

Diploid: Having two haploid chromosomal complements within a single nuclear envelope.

Directed mutagenesis: A process of altering the DNA sequence of at least one specific gene locus.

EDV (Essentially Derived Variety): A culture derived solely or predominantly from an initial strain or culture; a culture that has 75% or more of its genotype present in the genotype of an initial strain, that condition being a consequence of its derivation. Where derived directly or solely from the initial strain or culture, the culture likely has all of the genotype of that initial culture.

Flesh Thickness: A ratio of the maximum distance between the top of the stem and the uppermost part of the cap, divided by the maximum distance across the cap, measured on a longitudinally bisected mushroom; typically averaged over many specimens; subjectively called ‘meatiness’.

Flush: A period of mushroom production within a cropping cycle, separated by intervals of non-production; the term flush encompasses the terms ‘break’ and ‘wave’ and can be read as either of those terms.

Fungus: A microorganism classified as a member of the Kingdom Fungi.

Gene editing: The process of changing a specific gene, typically via CRISPR-Cas9 or a similar enzyme system, wherein the sequence of a functional gene is changed to make it inactive. In other uses, new sequences (including genes) may be introduced to the genome.

Genealogical relationship: A familial relationship of descent from one or more progenitors, for example that between parents and offspring.

Genetic identity: The genetic information that distinguishes an individual, including representations of said genetic information such as, and including: genotype, genotypic fingerprint, genome sequence, genetic marker profile; “genetically identical”=100% genetic identity, “X % genetically identical”=having X % genetic identity etc.

Genotypic fingerprint: A description of the genotype at a defined set of marker loci; the known genotype.

Gill: Lamella; part of the mushroom, the hymenophore- and basidium-bearing structure.

Haploid: Having only a single complement of nuclear chromosomes; see homokaryon.

Heteroallelic: Having two different alleles at a locus; analogous to heterozygous.

Heteroallelism: Differences between homologous chromosomes in a heterokaryotic genotype; analogous to heterozygosity.

Heterokaryon: As a term of art this refers to a sexual heterokaryon: a culture which has two complementary (i.e., necessarily heteroallelic at the Mat locus) types of haploid nuclei in a common cytoplasm, and is thus functionally and physiologically analogous to a diploid individual (but cytogenetically represented as N+N rather than 2N), and which is reproductively competent (in the absence of any rare interfering genetic defects at loci other than Mat), and which exhibits vegetative incompatibility reactions with other heterokaryons; also called a strain or stock in the strain development context.

Heterokaryon compatibility: The absence of antagonism observed during physical proximity or contact between two heterokaryons that are not genetically identical; see Heterokaryon Incompatibility.

Heterokaryon incompatibility: The phenomenon of antagonism observed during physical proximity or contact between two heterokaryons that are not genetically identical; a multilocus self/non-self-recognition system; i.e., a genetic system that allows one heterokaryon culture to discriminate and recognize another culture as being either self or non-self, that operates in basidiomycete heterokaryons to limit anastomosis (hyphal fusion) and cytoplasmic contact; vegetative incompatibility.

Heterokaryotic: Having the character of a heterokaryon.

Heteromixis: Life cycle involving mating between two different non-sibling haploid individuals or gametes; analogous to outbreeding.

Homoallelic: Having not more than one allele at a locus. The equivalent term in a diploid organism is ‘homozygous’. Haploid lines are by definition entirely homoallelic at all non-duplicated loci.

Homokaryon: A haploid culture with a single type (or somatic lineage) of haploid nucleus (cytogenetically represented as N), and which is ordinarily reproductively incompetent, and which does not exhibit typical self/non-self-incompatibility reactions with heterokaryons, and which may function as a gamete in sexually complementary anastomoses; a ‘line’ which, as with an inbred plant line, transmits a uniform genotype to offspring; a predominantly homoallelic line that mates well and fruits poorly is a putative homokaryon for strain development purposes; see discussion below.

Homokaryotic: Having the character of a homokaryon; haploid.

Hybrid: Of biparental origin, usually applied to heterokaryotic strains and cultures produced in controlled matings.

Hybridizing: Physical association, for example on a petri dish containing a sterile agar-based nutrient medium, of two cultures, usually homokaryons, in an attempt to achieve anastomosis, plasmogamy, and formation of a sexual heterokaryon (=mating); succeeding in the foregoing.

Hyphae: Threadlike elements of mycelium, composed of cell-like compartments.

Inbreeding: Matings that include sibling-line matings, back-matings to parent lines or strains, and intramixis; reproduction involving parents that are genetically related.

Incompatibility: See heterokaryon incompatibility.

Induced mutagenesis: A non-spontaneous process of altering the DNA sequence of at least one gene locus.

Initial culture: A culture which is used as starting material in a strain development process; more particularly a strain from which an Essentially Derived Variety is obtained.

Inoculum: A culture in a form that permits transmission and propagation of the culture, for example onto new media; specialized commercial types of inoculum include spawn and CI.

Intramixis: A uniparental sexual life cycle involving formation of a complementary ‘mated’ pair of postmeiotic nuclei within the basidium or individual spore.

Introgressive trait conversion: mating offspring of a hybrid to a parent line or strain such that a desired trait from one strain is introduced into a predominating genetic background of the other parent line or strain.

Lamella: see ‘gill’.

Line: A culture used in matings to produce a hybrid strain; ordinarily a homokaryon which is thus homoallelic, otherwise a non-heterokaryotic (non-NSNPP) culture which is highly homoallelic; practically, a functionally homokaryotic and entirely or predominantly homoallelic culture; analogous in plant breeding to an inbred line which is predominantly or entirely homozygous.

Lineage group: see ‘derived lineage group’. The set of EDVs derived from a single initial strain or variety.

Locus: A defined contiguous part of the genome, homologous although often varying among different genotypes; plural: loci.

Marker assisted selection: Using linked genetic markers including molecular markers to track trait-determining loci of interest among offspring and through pedigrees.

MAT: The mating-type locus, which determines sexual compatibility and the heterokaryotic state.

Mating: The sexual union of two cultures via anastomosis and plasmogamy; methods of obtaining matings between mushroom cultures are well known in the art.

Mycelium: The vegetative body or thallus of the mushroom organism, comprised of threadlike hyphae.

Mushroom: The reproductive structure of an agaric fungus; an agaric; a cultivated food product of the same name.

Neohaplont: A haploid culture or line obtained by physically deheterokaryotizing (reducing to haploid components) a heterokaryon: a somatically obtained homokaryon.

Offspring: Descendants, for example of a parent heterokaryon, within a single generation; most often used to describe cultures obtained from spores from a mushroom of a strain.

Outbreeding: Mating among unrelated or distantly related individuals; analogous to heteromixis in mushrooms.

Parent: An immediate progenitor of an individual; a parent strain is a heterokaryon, a parent line is a homokaryon; a heterokaryon may be the parent of an F1 heterokaryon via an intermediate parent line.

Pedigree-assisted strain development: The use of genealogical information to identify desirable combinations of lines in controlled mating programs.

Phenotype: Observable characteristics of a strain or line as expressed and manifested in an environment.

Plasmogamy: Establishment, via anastomosis, of cytoplasmic continuity leading to the formation of a sexual heterokaryon.

Progenitor: Ancestor, including parent (the direct progenitor).

Selfing: Mating among sibling lines; also intramixis.

Sexual compatibility: A condition among different lines of allelic non-identity at the Mat locus, such that two lines are able to mate to produce a stable and reproductively competent heterokaryon.

The opposite condition, sexual incompatibility, occurs when two lines have the same allele at the Mat locus.

Somatic: Of the vegetative mycelium.

Spawn: A mushroom culture, typically a pure culture of a heterokaryon, typically on a sterile substrate which is friable and dispersible particulate matter, in some instances cereal grain; commercial inoculum for compost; reference to spawn includes reference to the culture on a substrate.

Spore: Part of the mushroom, the reproductive propagule.

Stem: Stipe; part of the mushroom, the cap-supporting structure.

Sterile Growth Media: Nutrient media, sterilized by autoclaving or other methods, that support the growth of the organism; examples include agar-based solid nutrient media such as Potato Dextrose Agar (PDA), nutrient broth, and many other materials.

Stipe: see ‘stem’.

Strain: A heterokaryon with defined characteristics or a specific identity or ancestry; analogous to a variety.

Targeted mutagenesis: A process of altering the DNA sequence of at least one specific gene locus.

Tissue culture: A de-differentiated vegetative mycelium obtained from a differentiated tissue of the mushroom.

Trait conversion: A method for the selective introduction of the genetic determinants of one (a single-locus conversion) or more desirable traits into the genetic background of an initial strain while retaining most of the genetic background of the initial strain. See ‘Introgressive trait conversion’ and ‘Transformation’.

Transformation: A process by which the genetic material carried by an individual cell is altered by the incorporation of foreign (exogenous) DNA into its genome; a method of obtaining a trait conversion including a single-locus conversion.

Vegetative compatibility: The absence of the phenomenon of antagonism observed during physical proximity or contact between two heterokaryons that are not genetically identical, determined by a multilocus self/non-self recognition system that operates in basidiomycete heterokaryons to limit anastomosis (hyphal fusion) and cytoplasmic contact; Heterokaryon compatibility; the opposite of Vegetative incompatibility.

Vegetative incompatibility: The phenomenon of antagonism observed during physical proximity or contact between two heterokaryons that are not genetically identical, determined by a multilocus self/non-self recognition system that operates in basidiomycete heterokaryons to limit anastomosis (hyphal fusion) and cytoplasmic contact; Heterokaryon incompatibility.

Virus-breaking: Using multiple incompatible strains, i.e. strains exhibiting heterokaryon incompatibility, successively in a program of planned strain rotation within a mushroom production facility to reduce the transmission of virus from on-site virus reservoirs into newly planted crops.

Whole Genome Sequence (WGS): The DNA sequence of an organism, such as Agaricus bisporus.

Yield: The net fresh weight of the harvest crop, normally expressed in pounds per square foot.

Yield pattern: The distribution of yield within each flush and among all flushes; influences size, quality, picking costs, and relative disease pressure on the crop and product.

With respect to the definition of homokaryon above, it is noted that homokaryons and homoallelic lines are subject to technical and practical considerations: A homokaryon in classical terms is a haploid culture which is axiomatically entirely homoallelic. In practical terms, for fungal strain development purposes, the definition is broadened somewhat to accommodate both technical limitations and cytological variation, by treating all predominately homoallelic lines as homokaryons. Technical limitations include the fact that genomes contain duplicated DNA regions including repeated elements such as transposons and may also include large duplications of chromosomal segments due to historical translocation events; such regions may appear not to be homoallelic by most genotyping methods. Two different A. bisporus genomes sequenced by the Joint Genome Institute, a U.S. federal facility, differ in estimated length by 4.4%, and in gene numbers by 8.2%, suggesting a considerable amount of DNA duplication or rearrangement within different strains of the species. No presently available genome of A. bisporus can completely account for the physical arrangement of such elements and translocations, and so the assembled genome sequences of haploid lines may have regions that appear to be heteroallelic using currently available genotyping methods. Cytologically, a homokaryotic offspring will ordinarily be a spore that receives one haploid, postmeiotic nucleus. However, a spore receiving two third-division nuclei from the basidium will be genetically equivalent to a homokaryon. A spore receiving two second-division ‘sister’ postmeiotic nuclei will be a functional homokaryon even though some distal ‘islands’ of heteroallelism may be present due to crossovers during meiosis. Also, a meiosis that has an asymmetrical separation of homologues can produce an aneuploid, functionally homokaryotic spore in which an extra chromosome, producing a region of heteroallelism, is present. All of these cultures are highly homoallelic and all function as homokaryons. Technological limitations make it impractical to distinguish among such cultures, and also to rule out DNA segment duplication as an explanation for limited, isolated regions of the genome sequence assembly that appear to be heteroallelic. Therefore, in the present application, the use of the term ‘homoallelic’ to characterize a line includes entirely or predominately homoallelic lines, and cultures described in this way are functional homokaryons, are putatively homokaryotic, and are all defined as homokaryons in the present application.

Cultures of strains descended from lines B18287-s82 and/or WBL-s290, such as J19109, are noted to produce mushrooms, parts of mushrooms, parts of the culture, and strains and lines descended or derived from such cultures. Thus, the present invention encompasses cultures and parts of cultures of line B18287-s82 and WBL-s290, and F1 hybrid strain J19109, mushrooms and parts of mushrooms, including spores, produced therefrom. Additionally, EDVs and cultures derived solely or predominantly from an initial culture derived from F1 descendants of B18287-s82 and/or WBL-s290, dormant or active growing cultures present in dormant or germinating spores of strain J19109, and cultures incorporating the genetic material of an F1 descendant of strain J19109.

The present invention further relates to methods of making and using the strain J19109 and Essentially Derived Varieties (EDVs) of the strain J19109. Uses of cultures derived from J19109 and other cultures noted above include their incorporation into commercial products such as mushroom spawn and casing inoculum, as well as for the production of mushrooms, the development of additional novel cultures of A. bisporus, and for crop diversification and farm hygiene including ‘virus breaking.’

Now, with respect to the invention and as noted hereinabove, the present invention relates further to F1 strain J19109, homokaryotic lines thereof, and F2 hybrid derivatives thereof, such as crosses (i.e., matings) made between J19109 SSI homokaryon descendants and other white homokaryons, such as J11500-s80. Additionally, the invention includes cultures derived or descended from strain J19109. Such cultures may be used to produce mushrooms and parts of mushrooms.

The mating details are described herein with respect to J19109. In particular, one particular line J19109-s40 is mated with J11500-s80 to produce hybrid strain J20176. A representative culture of the strain J20176 is deposited under NRRL Accession No. \hat{ } \hat{ } \hat{ } \hat{ } \hat{ }. In one embodiment, the method further includes growing a crop of edible mushrooms by carrying out the steps described hereinabove. In another embodiment, the method may include using strain J20176 or Essentially Derived Varieties of strain J20176 in crop rotation to reduce pathogen pressure and pathogen reservoirs in mushroom growing facilities as described hereinabove. In yet another embodiment, the method includes using strain J20176 and EDVs of strain J20176 to produce offspring as described hereinabove.

Cultures of strains descended from lines J19109-s40 and J11500-s80, such as J20176, are noted to produce mushrooms, parts of mushrooms, parts of the culture, and strains and lines descended or derived from such cultures. Thus, the present invention encompasses cultures and parts of cultures of line J19109-s40 and J11500-s80, and hybrid strain J20176, mushrooms and parts of mushrooms, including spores, produced therefrom. Additionally, EDVs and cultures derived solely or predominantly from an initial culture derived from descendants of J10109-s40 and/or J11500-s80, dormant or active growing cultures present in dormant or germinating spores of strain J20176, and cultures incorporating the genetic material of a descendant of strain J20176. The present invention further relates to methods of making and using the strain J20176 and Essentially Derived Varieties (EDVs) of the strain J20176. Uses of cultures derived from J20176 and other cultures noted above include their incorporation into commercial products such as mushroom spawn and casing inoculum, as well as for the production of mushrooms, the development of additional novel cultures of A. bisporus, and for crop diversification and farm hygiene including ‘virus breaking.’

The morphological and physiological characteristics of strain J20176 in culture on Difco brand PDA medium, which is a standard culture medium, are provided as follows. Strain J20176 growing on PDA medium in an 8.5 cm diameter Petri dish produced a white or light brown-yellow or ‘tan’ colored irregularly lobate colony with a roughly circular overall outline that increased in diameter by (1.32-1.48-) 1.49 (−1.50-1.57) mm/day during dynamic equilibrium-state growth between days 8 and 26 after inoculation using a 3.5 mm diameter circular plug of the culture on PDA as inoculum. The strain has been increased by transfer of pure inocula into larger volumes of sterile culture media. No variant traits have been observed or are expected in strain J20176.

Methods for obtaining, manipulating, and mating cultures of the present invention, for producing offspring, inoculum, products, and crops of the current invention, for using a strain rotation program to improve mushroom farm hygiene, and for obtaining the genotypic fingerprint of mushroom cultures, are described hereinabove, and are also well known to practitioners of the art. Although the invention has been described in terms of particular embodiments in this application, one of ordinary skill in the art, in light of the teachings herein, can generate additional embodiments and modifications without departing from the spirit of, or exceeding the scope of, the claimed invention. Accordingly, it is understood that the descriptions herein are proffered only to facilitate comprehension of the invention and should not be construed to limit the scope thereof.

The J20176 hybrid strain is a mating made between J19109-s40 and J11500-s80. The J2076 hybrid demonstrably lacks the AI trait (see Table IV below). In addition, strain J20176 has a strong combination of desirable traits derived from the J15987 pedigree. Strain 20176 was grown in pre-commercial tests in North America, Europe and China, and has strong commercial potential.

Hybridization of Agaricus bisporus cultures of the invention may be accomplished by allowing two different cultures, one of which is a genetic line present in a spore of strain J19109, to grow together in close proximity, preferably on sterile media, until anastomosis (i.e., hyphal or cell fusion) occurs. In a successful mating, the resultant fusion culture is a first-generation outbred hybrid culture incorporating a genome from a line obtained from strain J19109. This line may have been obtained from a mushroom spore of J19109, which is one part of one embodiment of the present invention. Alternatively, this line may have been obtained from protoplasts derived from basidia, or other parts of the organism, of J19109, which is another part of one embodiment of the present invention.

For the purpose of this invention, the whole genomic DNA sequence of strain J19109 and its constituent homokaryons (B18287-s82 and WBL-s290) have been obtained. Also, the genomic DNA sequence of lines J11500-s80 and J19109-s40 (parents of J20176) have also been obtained. For DNA preparation, cultures were grown in sterile broth growth medium after maceration. After 2-4 weeks, hyphal cells were collected by filtration, were frozen at −80C, and were lyophilized until dry. Cap tissue was obtained from mushrooms produced by cultures of the heterokaryotic strain (such as J20176) and was frozen and lyophilized. DNA was extracted from the lyophilized samples using a CTAB protocol followed by RNAse treatment and gel purification. A contractor, Genewiz (New Brunswick, New Jersey) prepared DNA libraries from the DNA of each culture and sequenced the libraries using Illumina technology. Assemblies of the reads into genomic sequence using the public-domain reference genome sequence of H97 version 2.0 (Morin et al. 2012; PNAS 109 (43): 17501, included herein as a reference) was performed by the Sylvan, Inc. Consequently about 93% to about 95% of the entire genotype of strain J20176 and its parental homokaryons are known to Sylvan, Inc. with certainty.

The industry's understanding of the genetic control of heterokaryon incompatibility is limited; however, it is known that it is separate from the well-known mating-type gene or genes, and that more than one gene is involved, for example 4 or 5 in Coprinopsis, with the chromosomal locations undefined.

In the 2010s, powerful techniques based on SNP analysis became available. Each SNP marker represents a difference in a single DNA building block, termed a nucleotide. For example, in a mutation, an ‘A’ can become a ‘G’. Furthermore, large scale, greater than 50 times genome coverage may be obtained through Whole Genome Sequence (WGS) technologies, most notably using equipment manufactured by Illumina. These “Illumina reads” can then be aligned onto a Reference Genome, using appropriate software. The Reference Genome is important because the precise location of each SNP in the genome can be defined numerically, with a base pair position.

In Table I, an SNP-based comparison of the genotypes of cultures used to make the Invention described herein is provided. To produce whole genome sequence, we utilized Illumina 250 bp reads aligned to the H97 version 2.0 reference genome using DNASTAR's Lasergene version 18. The genotype of strains J19109 and J20176 and their parental lines B18287-s82 and WBL-s290 (for strain J19109) and J11500-s80 and J19109-240 (for strain J20176) at numerous sequence-characterized marker loci distributed at intervals along each of the 19 H97 Version 2.0 reference scaffolds larger than 100 Kbp in length is provided in Table I.

TABLE I
SNP markers and positions in breeding stocks

| | H97 v | | | | | | |
|---|---|---|---|---|---|---|---|
| Scaf- | 2.0 | | B18287- | | | J11500- | J19109- |
| fold | coord. | H97 | s82 | WBL-s290 | J19109 | s80 | s40 |
| 1 | 99995 | CTACATTGA | CTACGTTGA | CTACATTGA | CTACrTTGA | CTACGTTGA | CTACATTGA |
| 1 | 349966 | AAGGTGGTT | AAGGCGGTT | AAGGTGGTT | AAGGyGGTT | AAGGCGGTT | AAGGTGGTT |
| 1 | 600145 | GTTGGATTA | GTTGGCTTA | GTTGGATTA | GTTGGmTAA | GTTGrATTA | GTTGGATTA |
| 1 | 850017 | CCTTTTCAC | CTTTTTCGC | CCTTTTCAC | CyTTTTCrC | CTTTTTCGC | CCTTTTCAC |
| 1 | 1099971 | GTCGACACC | GTCGACACC | GTCGACACC | GTCGACACC | GTCGGCACC | GTCGACACC |
| 1 | 1350278 | GGAGAGTCG | GGAGGTTCG | GGAGAGTCG | GGAGrkTCG | GGAGGTTCG | GGAGAGTCG |
| 1 | 1599956 | AATAAGCGC | AATAAGCGC | AATAAGCGC | AATAAGCGC | AATAGGCGC | AATAAGCGC |
| 1 | 1869790 | CCGTGTATC | CCGTGTATC | CCGTGTATC | CCGTrTATC | CGAGCAATT | CCGTGTATC |
| 1 | 2119049 | ACAATCCAA | ACAATCCAA | ACAATCCAA | ACAAyyCAA | ACAACTCAA | ACAATCCAA |
| 1 | 2360610 | TTCTACCAC | TTCTACCAC | TTCTACCAC | TTCTACCAC | TTCTGCCAC | TTCTACCAC |
| 1 | 2612870 | AATAGGAGT | AATAGGAGT | AATAGGAGT | AATAGGAGT | AATAAGAGT | AATAGGAGT |
| 1 | 2804522 | GAAGACGAC | GAAGGCGAC | GAAGACGAC | GAAGrCGAC | GAAGGGGAC | GAAGACGAC |
| 1 | 2858975 | GCCGTTCTT | GCCGCTCTT | GCCGTTCTT | GCCGyTCTT | GCCGCTCTT | GCCGTTCTT |
| 1 | 3069801 | CCAAACGCG | CCAAGCGCG | CCAAACGCG | CCAArCGCG | CCAAGCGCG | CCAAACGCG |
| 1 | 3256057 | TATCTGTTT | TATCCGTTT | TATCTGTTT | TATCyGTTT | TATCCGTTT | TATCTGTTT |
| 2 | 101820 | ATTAAAGAT | ATCAAAGAT | ATTAAAGAT | ATyAAAGAT | ATTAAAGAT | ATTAAAGAT |
| 2 | 350156 | TCGGGGGTG | TCGGAGGTG | TCGGGGGTG | TCGGrGGTG | TCGGGGGTG | TCGGGGGTG |
| 2 | 600112 | ATGTATACG | ATGTGTACG | ATGTATACG | ATGTrTACG | ATGTATACG | ATGTATACG |
| 2 | 850338 | TGGTGCTAA | TGGTGCTAA | TGGTGCTAA | TGGTGCTAA | TGGTGCTAA | TGGTGCTAA |
| 2 | 1099413 | CCTGACTCA | CCTGGCTCA | CCTGACTCA | CCTGrCTCA | CCTGACTCA | CCTGACTCA |
| 2 | 1349512 | CTCAGCAGT | CTCAACGGT | CTCAGCAGT | CTCArCrGT | CTCAGCAGT | CTCAGCAGT |
| 2 | 1600085 | CACAATGCC | CACAATGCC | CACAATGCC | CACAATGCC | CACAATGCC | CACAATGCC |
| 2 | 1902928 | GATGGATGT | GATGGATGT | GATGGATGT | GATGGATGT | GATGGATGT | GATGGATGT |
| 2 | 2150201 | GTCGTAGGT | GTCGAAGGT | GTCGTAGGT | GTCGwAGGT | GTCGTAGGT | GTCGTAGGT |
| 2 | 2400354 | CAGAGTCGC | CAGAGTCGC | CAGAGTCGC | CAGAGTCGC | CAGAGTCGC | CAGAGTCGC |
| 2 | 2650136 | ATAATTCCT | ATAAATCCT | ATAATTCCT | ATAAwTCCT | ATAATTCCT | ATAATTCCT |
| 2 | 2903045 | AGAAATAGA | AGAAATAGA | AGAAATAGA | AGAAATAGA | AGAAATAGA | AGAAATAGA |
| 2 | 3048019 | GTCCGCTGC | GTCCACTGC | GTCCGCTGC | GTCCrCTGC | GTCCGCTGC | GTCCGCTGC |
| 3 | 57118 | TATAGCAGC | TATAGCAGC | TATGACAGC | TATrrCAGC | TATAGCAGC | TATAGCAGC |
| 3 | 118150 | GTTTGTCCT | GTTTATCCT | GTTTGTCCT | GTTTrTCCT | GTTTGTCCT | GTTTATCCT |
| 3 | 131389 | AGACCGGCG | AGACCGGCG | AGACCGGCG | AGACCGGCG | AGACCGGCG | AGACCGGCG |
| 3 | 175472 | CTTTATTTC | CTTTATTTC | CTTTATTTC | CTTTATTTC | CTTTATTTC | CTTTATTTC |
| 3 | 250112 | GCAGGAGAG | GCCGAAGAG | GCAGGAGAG | GCmGrAGAG | GCAGGAGAG | GCCGAAGAG |
| 3 | 379203 | ATAGCGGAA | ATAGAGGAA | ATAGCGGAA | ATAGmGGAA | ATAGCGGAA | ATAGAGGAA |
| 3 | 614937 | CAAAATCTG | CAAACTCTG | CAAAATCTG | CAAAmTCTG | CAAAATCTG | CAAACTCTG |
| 3 | 750074 | GTTCTTTTC | GTTCATTTC | GTTCTTTTC | GTTCwTTTC | GTTCTTTTC | GTTCATTTC |
| 3 | 1126997 | TCAAAGGCG | TCAAGGGCG | TCAAAGGCG | TCAArGGCG | TCAAAGGCG | TCAAGGGCG |
| 3 | 1250161 | AGTCTCCTT | AGTCCCCTT | AGTCTCCTT | AGTCyCCTT | AGTCTCCTT | AGTCCCCTT |
| 3 | 1296141 | ATCGGTCAT | ATCGGTCAT | ATCGGTCAT | ATCGGTCAT | ATCGGTCAT | ATCGGTCAT |
| 3 | 1510819 | CCACTGATT | CCACAGATT | CCACTGATT | CCACwGATT | CCACTGATT | CCACAGATT |
| 3 | 1774892 | CCGTATGGG | CCGTGTGGG | CCGTATGGG | CCGTrTGGG | CCGTATGGG | CCGTGTGGG |
| 3 | 2008438 | AGCATAGCC | AGCAGAGCC | AGCATAGCC | AGCAkAGCC | AGCATAGCC | AGCAGAGCC |
| 3 | 2250000 | CGTGGCGAT | CGTGGCAAT | CGTGGCGAT | CGTGGCrAT | CGTGGCGAT | CGTGGCAAT |
| 3 | 2274053 | AAACCAAGA | AAACCAAGA | AAACCAAGA | AAACCAAGA | AAACCAAGA | AAACCAAGA |
| 3 | 2384173 | TGACCAAGC | TGACCAAGC | TGACCAAGC | TGACCAAGC | TGACCAAGC | TGACCAAGC |
| 3 | 2520748 | TAATTCCAC | TAATTCCAC | TAATTCCAC | TAATTCCAC | TAATTCCAC | TAATTCCAC |
| 3 | 2523207 | CAGTCCATA | CAGTCCATA | CAGTCCATA | CAGTCCATA | CAGTCCATA | CAGTCCATA |
| 4 | 100004 | GAGTGATAA | GAGTGATAA | GAGTAATGA | GAGTrATrA | GAGTGATAA | GAGTGATAA |
| 4 | 383799 | CAGCCAGAC | CCGCAAGAC | CAGCAAGAC | CmGCAAGAC | CAGCCAGAC | CCGCAAGAC |
| 4 | 598147 | GATCGACAG | GATCAACAG | GATCAACAG | GATCAACAG | GATCGACAG | GATCAACAG |
| 4 | 852119 | CGAATATTC | CGAACACTC | CGAACACTC | CGAACACTC | CGAATATTC | CGAACACTC |
| 4 | 1100085 | GATGCCGAA | GATGACGAA | GATGACGAA | GATGACGAA | GATGCCGAA | GATGACGAA |
| 4 | 1350536 | CGAACTCGG | CGAAACCGG | CGAAACCGG | CGAAACCGG | CGAACTOGG | CGAAACCGG |
| 4 | 1599885 | GATACTTGC | GATACTTGC | GATAATTGC | GATAmTTGC | GATACTTGC | GATACTTGC |
| 4 | 1850288 | ATTCGTGTA | ATTCACGTA | ATTCACGTA | ATTCACGTA | ATTCGTGTA | ATTCACGTA |
| 4 | 2100356 | TCAGAGACC | TCAGGGACC | TCAGAGACC | TCAGrGACC | TCAGGGACC | TCAGGGACC |
| 4 | 2284257 | TCTGGACTG | TCTGGACTG | TCTGGACTG | TCTGGACTG | TCTGAACTG | TCTGGACTG |
| 5 | 100211 | TCCTTGAAT | TCCTCGAAT | TCCTTGAAT | TCCTyGAAT | TCCTCGAAT | TCCTTGAAT |
| 5 | 350872 | GGCGTGCCC | GGCGTGCCC | GGCGTGCCC | GGCGTGCCC | GGCGCGCCC | GGCGTGCCC |
| 5 | 599922 | CGTCATTCA | CGTCATTCA | CGTCATTCA | CGTCATTCA | CGTCGTTCA | CGTCATTCA |
| 5 | 851262 | TAATTCTCT | TAATCGTCT | TAATTCTCT | TAATysTCT | TAATCGTCT | TAATTCTCT |
| 5 | 1099776 | ACATTGACA | ACATTGACA | ACATTGACA | ACATyGACA | ACATCGACA | ACATTGACA |
| 5 | 1352539 | TTGTGATCC | TTGTGGTCC | TTGTGATCC | TTGTGrTCC | TTGTTGTCC | TTGTGATCC |
| 5 | 1599904 | AACTTCCTT | AACTCCCTT | AACTTCCTT | AACTyCCTT | AACTCCCTT | AACTTCCTT |
| 5 | 1851458 | AAATAATCC | AAATAATCC | AAATAATCC | AAATAATCC | AAATTCTCC | AAATAATCC |
| 5 | 2100025 | CCCTTAGTC | CCCTTAGTC | CCCTTAGTC | CCCTTAGTC | CCCTCAGTC | CCCTTAGTC |
| 5 | 2278878 | GGTCGAAAA | GGTCGAAAA | GGTCGAAAA | GGTCGAAAA | GGTCAAAAA | GGTCGAAAA |
| 6 | 116552 | CCTTGTCGG | CCTTGTCGG | CCTTGTCGG | CCTTGTCGG | CCTGATCGG | CCTTGTCGG |
| 6 | 350337 | CATTTGGTT | CATTTGGTT | CATTTGGTT | CATTTGGTT | CATTCGGTT | CATTTGGTT |
| 6 | 600047 | GGAGCATTT | GGAGCATTT | GGAGCATTT | GGAGCATTT | GGAGTATTT | GGAGCATTT |
| 6 | 849990 | AGTTCAGGA | AGTTCAGGA | AGTTCAGGA | AGTTCAGGA | AGTTTAGGA | AGTTCAGGA |
| 6 | 1098535 | CAAAGATTG | CAAAGATTG | CAAAGATTG | CAAAGATTG | CAAAAATTG | CAAAGATTG |
| 6 | 1349453 | TGTCGGTAG | TGTCGGTAG | TGTCGGTAG | TGTCGGTAG | TGTCAATAG | TGTCGGTAG |
| 6 | 1600000 | AAACCTGGA | AAACCTGGA | AAACCTGGA | AAACCTGGA | AAACCTGGA | AAACCTGGA |
| 6 | 1764645 | AACCGGATT | AACCGGATT | AACCGGATT | AACCGGATT | AACCAGATT | AACCGGATT |
| 6 | 2000087 | GATTTTGCG | GATTTTGCG | GATTTTGCG | GATTTTGCG | GATTCTGCG | GATTTTGCG |
| 6 | 2252662 | GGGTTGGTA | GGGTCGGTA | GGGTTGGTA | GGGTyGGTA | GGGTCGGTA | GGGTCGGTA |
| 7 | 122800 | GTCGCGCAA | GTCGAGCAA | GTCGCGCAA | GTCGmGCAA | poor depth | GTCGCGCAA |
| 7 | 227441 | ACACATACT | ACACATACT | ACACGTACT | ACACrTACT | ACACATACT | ACACGTACT |
| 7 | 350044 | ATATTCTTT | ATATTCTTT | ATATCCTTT | ATATyCTTT | ATATTCTTT | ATATCCTTT |
| 7 | 600111 | CAATTATTA | CAATTATTA | CAATCATTA | CAATyATTA | CAATTATTA | CAATCATTA |
| 7 | 850516 | TGACGCATA | TGACGCATA | TGACACATA | TGACrCATA | TGACGCATA | TGACACATA |
| 7 | 1100248 | TCACGGAAG | TCACGGAAG | TCACAGAAG | TCACrGAAG | TCACGGAAG | TCACAGAAG |
| 7 | 1350089 | CTTTTCCCC | CTTTTCCCC | CTTTCCCCC | CTTTyCCCC | CTTTTCCCC | CTTTCCCCC |
| 7 | 1605047 | ATACTTGGC | ATACTTGGC | ATACGTGAC | ATACkTGrC | ATACTTGGC | ATACGTGAC |
| 7 | 1850000 | GAGATACT | GAGATACT | GAGATACT | GAGATACT | GAGATACT | GAGATACT |
| 7 | 1898793 | TCCGCATAA | TCCGCATAA | TCCGTATGA | TCCGyATrA | TCCGCATAA | TCCGTATGA |
| 7 | 1991505 | TCTACGGTT | TCTACGGTT | TCTAAAGTT | TCTAmrGTT | TCTACGGTT | TCTAAAGTT |
| 8 | 350000 | ATTGACGCG | ATTGACGCG | ATTGACGCG | ATTGACGCG | ATTGACGCG | ATTGACGCG |
| 8 | 606991 | GTGTATTCT | GTGTATTCT | GTGTATTCT | GTGTATTCT | GTGTATTCT | GTGTATTCT |
| 8 | 610549 | GGAACTTGA | GGAACTTGA | GGAACTTGA | GGAACTTGA | GGAACTTGA | GGAACTTGA |
| 8 | 834519 | ACACATAGA | ACACATAGA | ACACATAGA | ACACATAGA | ACACATAGA | ACACATAGA |
| 8 | 1100000 | CATACGATC | CATACGATC | CATACGATC | CATACGATC | CATACGATO | CATACGATO |
| 8 | 1350240 | ACGGGTACT | ACGGGTACT | ACGGGTACT | ACGGGTACT | ACGGGTACT | ACGGGTACT |
| 8 | 1354068 | AGAATGCCT | AGAATGCCT | AGAATGCCT | AGAATGCCT | AGAATGOCT | AGAATGOCT |
| 8 | 1614036 | TTATCAGTA | TTATCAGTA | TTATCAGTA | TTATCAGTA | TTATCAGTA | TTATCAGTA |
| 8 | 1869238 | TGGAGGTTG | TGGAGGTTG | TGGAGGTTG | TGGAGGTTG | TGGAGGTTG | TGGAGGTTG |
| 9 | 100105 | CTCAACCGA | CTCAACCGA | CTCAACCGA | CTCAACCGA | CTCAACCGA | CTCAACCGA |
| 9 | 352455 | AGTCCTCCA | AGTCCTCCA | AGTCCTCCA | AGTCCTCCA | AGTCCTCCA | AGTCCTCCA |
| 9 | 661330 | TAGATTAAC | TAGAGTAAC | TAGATTAAC | TAGAkTAAC | poor depth | TAGATTAAC |
| 9 | 800528 | TCGACGACC | TCGACGACC | TCGACGACC | TCGACGACC | TCGACGACC | TCGACGACC |
| 9 | 1010845 | GGGTGGTGA | GGGTGGTGA | GGGTGGTGA | GGGTGGTGA | GGGTGGTGA | GGGTGGTGA |
| 9 | 1090163 | GAATATCCA | GAATATCCA | GAATGTCCA | GAATrTCCA | poor depth | GAATGTCCA |
| 9 | 1335069 | ATTTGCTTC | ATTTACTTC | ATTTGCTTC | ATTTrCTTC | ATTTGCTTC | ATTTGCTTC |
| 9 | 1656962 | TATCTACTG | TATCTACTG | TATCTACTG | TATCTACTG | TATCTACTG | TATCTACTG |
| 10 | 100438 | AATTAATTT | AATTAATTT | AATTAATTT | AATTAATTT | AATTCATTT | AATTAATTT |
| 10 | 352915 | GCGTTCGTG | GCGTCCGTG | GCGTTCGTG | GCGTyCGTG | GCGTCCGTG | GCGTTCGTG |
| 10 | 588452 | ATCCTCCAA | ATCCCCCAA | ATCCTCCAA | ATCCyCCAA | poor depth | ATCCTCCAA |
| 10 | 862520 | AAGATGAAC | AAGACGAAC | AAGATGAAC | AAGAyGAAC | poor depth | AAGATGAAC |
| 10 | 1110433 | GGAAGACAA | GGAAGACAA | GGAAGACAA | GGAAGACAA | GGAAAACAA | GGAAGACAA |
| 10 | 1303902 | TGATTTACT | TGATTTACT | TGATTTACT | TGATTTACT | TGATCTACT | TGATTTACT |
| 10 | 1490452 | AATCAGATG | AATCAGATG | AATCAGATG | AATCAGATG | AATCTGATG | AATCAGATG |
| 11 | 104770 | AATGAGAGG | AATGAGAGG | AATGAGAGG | AATGAGAGG | AATGAGAGG | AATGAGAGG |
| 11 | 349990 | GACGGCTTC | GACGGCTTC | GACGACTTC | GACGrCTTC | GACGGCTTC | GACGGCTTC |
| 11 | 600001 | TGGGCGCGC | TGGGCGCGC | TGGGAGCGC | TGGGmGCGC | TGGGCGCGC | TGGGCGCGC |
| 11 | 908344 | TAGAAAGAA | TAGAAAGAA | TAGACAGAA | TAGAmAGAA | TAGAAAGAA | TAGAAAGAA |
| 11 | 1100296 | TTCTAAAAT | TTCTAAAAT | TTCTGAAAT | TTCTrAAAT | TTCTAAAAT | TTCTAAAAT |
| 11 | 1239957 | GCTTACTGC | GCTTACTGC | GCTTGCTGC | GCTTrCTGC | GCTTACTGC | GCTTACTGC |
| 12 | 86057 | ACAAGTCAA | ACAAATCAA | ACAAGTCAA | ACAArTCAA | poor depth | ACAAATCAA |
| 12 | 113463 | CGAGACCTT | CGAGGCCTT | CGAGGCCTT | CGAGGCCTT | poor depth | CGAGGCCTT |
| 12 | 583507 | GCTTGCTGT | GCTTACTGT | GCTTGCTGT | GCTTrCTGT | poor depth | GCTTACTGT |
| 12 | 700059 | GCTGCCATG | GCTGTCATG | GCTGTCATG | GCTGTCATG | GCTGCCATG | GCTGTCATG |
| 12 | 1000704 | TTCTGGTGC | TTCTAGTGC | TTCTAGTGC | TTCTAGTGC | TTCTGGTGC | TTCTAGTGC |
| 13 | 100697 | ACGTCTTTA | ACGTATTTA | ACGTCCTTA | ACGTmCTTA | ACGTATTTA | ACGTCCTTA |
| 13 | 370910 | TTCGGGATG | TTCGGGATG | TTCGGGATG | TTCGGGATG | TTCGGGATG | TTCGGGATG |
| 13 | 604345 | CTTCAGCAT | CTTCCGCAT | CTTCAGCAT | CTTCmGCAT | CTTCCGCAT | CTTCAGCAT |
| 13 | 850249 | GGCTAGTAA | GGCTAGTAA | GGTTGGTGA | GGyTrGTrA | GGTTGGTGA | GGTTGGTGA |
| 14 | 113109 | AGGGAAATA | AGGGGAATA | AGGGGAATA | AGGGGAATA | AGGGAAATA | AGGGGAATA |
| 14 | 372086 | CGATCCCTT | CGATTCCTT | CGATTCTTT | CGATTCyTT | CGATCCCTT | CGATTCCTT |
| 14 | 725684 | ATGAGTTCG | ATGAGTTTG | ATGAATTTG | ATGArTTyG | ATGAGTTCG | ATGArTTyG |
| 15 | 97145 | TGACGTTTT | TGACATTTT | TGACATTTT | TGACATTTT | TGACGTTTT | TGACATTTT |
| 15 | 449866 | GAATTTCGG | GAATTTCGG | GAATCTCGG | GAATyTCGG | GAATCTCGG | GAATCTCGG |
| 16 | 208609 | CACATGCAC | CACACGCAC | CACATGCAC | CACAyGCAC | CACACGCAC | CACATGCAC |
| 16 | 400000 | CCTCGGATT | CCTCGGATT | CCTCGGATT | CCTCGGATT | CCTCGGATT | CCTCGGATT |
| 17 | 120000 | TATTCTTCA | TATTCTTCA | TATTCTTCA | TATTCTTCA | TATTCTTCA | TATTCTTCA |
| 17 | 338415 | TGAGAAGCC | TGAGAAGCC | TGAGAAGCC | TGAGAAGCC | TGAGAAGCC | TGAGAAGCC |
| 17 | 449833 | ATCAGACAA | ATCAAACTA | ATCAAACTA | ATCAAACTA | ATCAAACTA | ATCAAACTA |
| 18 | 101884 | ATTACGGAC | ATTACGGAC | ATTATGGAC | ATTAyGGAC | ATTACGGAC | ATTACGGAC |
| 19 | 98377 | GCTATTGGG | GCTATTGGG | GCTACTGGG | GCTAyTGGG | GCTATTGGG | GCTACTGGG |

It can clearly be seen that the SNP pattern for each of the lines and strains is unique and is therefore a robust form of genetic identification. Furthermore, anyone who is skilled in the Art will be able to verify the SNP markers described in Table I.

Data in Table I are displayed as 9-mers and in most cases the middle base (position 5) provides the SNP. We picked robust markers at loci aligned across each of the first 19 scaffolds. The majority of the markers in Table II are standard markers used in other patent cases, for example U.S. Pat. Nos. 9,017,988 and 10,440,930. We determined the SNP alleles at those loci for the homokaryotic parents of J20176 (J11500-s80 and J19109-s40), in addition to B18287-s82, WBL-s290, the J19109 heterokaryon, and J11500-s80.

TABLE II
Genotype of Six Standard Markers

| | H97 v | | | | | | |
|---|---|---|---|---|---|---|---|
| | 2.0 | | B18287- | WBL- | | J11500- | J19109- |
| Marker/Locus | coord. | H97 | s82 | s290 | J19109 | s80 | s40 |
| p1n150 (scaffold_1) | 868615 | 1T | 2A | 1T | 1T/2A | 2 | IT |
| ITS (scaffold_10) | 1612110 | I1 | I1 | I2 | I1/I4 | I4 | I2 |
| MFPC-ELF (scaffold_8) | 829770 | E1 | E1 | E1 | E1/E1 | E1 | E1 |
| AN (scaffold_9) | 1701712 | N1 | N1 | N1 | N1/N1 | N1 | N1 |
| AS (scaffold_4) | 752867 | SD | SD | SC | SC/SD | SD | SC |
| FF (scaffold_12) | 281674 | FF1 | FF2 | FF2 | FF2/FF2 | FF1 | FF2 |

Table II reports data from six genetic loci that are standardly reported for same panel of strains utilized in Table I, for example U.S. Pat. Nos. 9,017,988 and 10,440,930. As in Table II, there are two alleles at each marker locus for one heterokaryotic strain (J19109) and a single allele per locus for the six homokaryotic cultures. The data was prepared by the Applicant using targeted Polymerase Chain Reactions (PCR) to amplify genomic regions spanning the defined markers from each of the culture DNAs. PCR primers that bracket the defined marker regions, at locations indicated by the positional information provided below, were utilized to generate the data; methods of designing suitable primers are well known in the art. From the amplified PCR product, DNA was sequenced by a contractor, Eurofins (Louisville, Kentucky), using methods of their choice, and the genotypes were determined by direct inspection of these sequences in comparison to Sylvan's database of reference marker/allele sequences. In most cases the sequence was further confirmed by direct inspection of the corresponding whole genome sequence for that culture.

Description of the p1n150-G3-2 Marker:

The 5′ end of this marker segment begins at position 1 with the first “T” in the sequence TCCCAAGT, corresponding to H97 JGI V2.0 Scaffold 1 position 868615 (Morin et al. 2012) and extending in a reverse orientation (relative to the scaffold orientation) for ca. 600 nt in most alleles; an insertion in the DNA of allele 1T has produced a longer segment. At present, 9 alleles incorporating at least 30 polymorphic positions have been documented from diverse strains in Sylvan's culture collection. This marker is an important part of Agaricus breeding programs, because it is tightly linked to the MAT locus. MAT is a complex of genes that controls mating between homokaryons.

Allele 1T, as noted above contains a transposon of 320 base pairs. All other known alleles of p1n150 lack this intron.

Analysis with p1n150 of a panel of 88 homokaryotic SSIs from J19109 identified 52 matches with the p1n150 allele for Mat-1, and a second cohort of 36 strains which contained a different mating type allele, which was provisionally called “Mat-X”.

The complete set of Mat-X homokaryons were crossed to a panel of J11500 SSI homokaryons which were known to be MAT-2, in order to generate new crosses. To our surprise, none of these crosses were successful and it was concluded that MAT-X was functionally the same as MAT-2. The MAT-2 allele in J19109 must have descended from the wild W01-s1 homokaryon, because the other parental homokaryon in B18287, So76-s12b, was known to be MAT-1.

The next step was to align DNA from the p1n150 amplicon from W01-s1, B18187-s82 and J11500-s80, to see if the p1n50 sequences for MAT-2 were a complete match. Data showed that that there were differences between the sequence of J11500-s80 MAT-2 and the sequence possessed by W01-s1 and B18187-s82. The new p1n150 allele discovered in W01-s1 and B18287-s82 was therefore named “2A”.

Allele 1T: insertion of ABR transposon of 320 nt @206\hat{ }207, ‘A’@321; ‘T’@327; ‘C’@374; ‘G’@378: ‘G’@422; etc.

Allele 2: no Abr1 insertion; ‘A’@321; ‘C’@327, ‘C’@374; ‘C’@378; ‘G’@422; etc.

Allele 2A: no Abr1 insertion; ‘A’@321; ‘C’@327, ‘T’@374, ‘C’@378: ‘T’@422; etc.

### Description of the ITS (=ITS 1+2 Region) Marker:

The ITS segment is part of the nuclear rDNA region which is located on chromosome 9 (Scaffold 10 in JGI H97 V2.0). The rDNA is a cassette that is tandemly repeated up to an estimated 100 times in the haploid genome of A. bisporus. Therefore, there is no single precise placement of this sequence in the assembled H97 genome, and in fact it is difficult or impossible to precisely assemble the sequence over all the tandem repeats. Three cassette copies were included on scaffold 10 of the H97 JGI V2.0 assembly, beginning at position 1612110; a partial copy is also assembled into scaffold 29 (Morin et al. 2012). The 5′ end of this marker segment begins at position 1 with the first “G” in the sequence GGAAGGAT and extending in a forward orientation (relative to the scaffold orientation) for ca. 703-704 nt in most alleles. At present, more than 9 alleles incorporating at least 11 polymorphic positions have been documented from diverse strains in Sylvan's culture collection.

Alleles present in this Application are I1, I2 and I4. Characterized as follows (using the format: nucleotide base character @ alignment position, based on alignment of 9 alleles).
Allele I1: ‘C’@52; ‘T’@461; ‘T@522; ‘T’@563; etc.
Allele I2: ‘T’@52; ‘T’@461; ‘T’@522; ‘T’@563; etc.
Allele I4: ‘C’@52; ‘A’@461; ‘C’@522; ‘C’@563; etc.

### Description of the MFPC-1-ELF Marker:

The 5′ end of this marker segment begins at position 1 with the first “G” in the sequence GGGAGGGT, corresponding to H97 JGI V2.0 Scaffold 8 position 829770 (Morin et al. 2012) and extending in a forward orientation (relative to the scaffold orientation) for ca. 860 nt in most alleles. At present, at least 7 alleles incorporating at least 40 polymorphic positions have been documented from diverse strains in Sylvan's culture collection.

Note that MFPC-ELF is linked to the PPC-1 locus on Scaffold 8, which is known to be a major factor controlling cap color. Mushrooms with white cap color have the E1 allele at MFPC-ELF.
Allele E1: ‘A’@63; ‘A’@77; ‘A’@232; ‘A’@309; ‘T’@334, ‘A’@390; ‘A’@400; ‘T’@446, ‘A’@481; etc.

All strains in Table II shared the E1 allele at MFPC-ELF.

### Description of the AN Marker:

The 5′ end of this marker segment begins at position 1 with the first “G” in the sequence GGGTTTGT, corresponding to H97 JGI V2.0 Scaffold 9 position 1701712 (Morin et al. 2012) and extending in a forward orientation (relative to the scaffold orientation) for ca. 1660 (in the H97 genome) to 1700 nt (in alignment space) in known alleles; several insertions/deletions have created length polymorphisms which, in addition to point mutations of individual nucleotides, characterize the alleles. At present, 5 alleles incorporating more than 70 polymorphic positions have been documented from diverse strains in Sylvan's culture collection.

Only one Allele, N1 is present in the strains used in this Application (using the format: nucleotide base character @ alignment position, based on alignment of alleles N1 through N4):
Allele N1: ‘G’@640; [deletion]@844-846; ‘C’@954; ‘T’@882; ‘A’@954, etc.

All strains in this study shared the same N1 allele on scaffold 9.

### Description of the AS Marker:

The 5′ end of this marker segment begins at position 1 with the first “G” in the sequence GG(T/N)GTGAT, corresponding to H97 JGI V2.0 Scaffold 4 position 752867 (Morin et al. 2012) and extending in a forward orientation (relative to the scaffold orientation) for ca. 1620 (in the H97 genome) to 1693 nt (in alignment space) in known alleles; several insertions/deletions have created 30 length polymorphisms which, in addition to point mutations of individual nucleotides, characterize the alleles. At present, 7 alleles incorporating more than 80 polymorphic positions have been documented from diverse strains in Sylvan's culture collection.

Alleles present in the B18287/J19109 immediate pedigree are alleles SC and SD, characterized in part as follows (using the format: nucleotide base character @ alignment position, based on alignment of alleles SA through SG):
Allele SC: ‘T’@28; ‘GATATC’@258-263; ‘G’@275; [insertion]+‘TTTCTCAGC’+[insertion]@309-249; ‘C’@404, etc.
Allele SD: ‘C’@28; [deletion]@258-263; ‘T’@275; [deletion]@309-249; ‘T’@275; [deletion]@309-349, ‘T’@404, etc.

### Description of the FF Marker:

The 5′ end of this marker segment begins at position 1 with the first “T” in the sequence TTCGGGTG, corresponding to H97 JGI V2.0 Scaffold 12 position 281999 (Morin et al. 2012) and extending in a forward orientation (relative to the scaffold orientation) for ca. 570 nt in most alleles. At present, 7 alleles incorporating at least 20 polymorphic positions have been documented from diverse strains in Sylvan's culture collection.

Two alleles are found in the strains used to make this Invention; FF1 and FF2.
Allele FF1: ‘CCG’@48-50, ‘C’@91, etc.
Allele FF2: ‘TTC”@48-50, ‘C’@91, etc.

The FF1 genotype is found in white strains such as So76 and U1 and associated EDVs, such as A15. In this study, one of the breeding stocks, So76-s12b had inherited this marker.

Possession of the FF2 genotype, allows W0-s01, B18287-s82, WBL-s290, J19109 and J11500-s80 (and associated EDVs and direct descendants) to be identified.

As noted above, one of the goals of the J19109 and J20176 pedigrees were to develop a method to exclude the Aggressive Incompatibility (AI) trait while preserving as many of the positive traits seen in J15987 as possible. To test this concept, a test protocol was designed to measure the effects of mixing small quantities of spawn from F2 hybrid strains having a line from J19109 as one homokaryon parent and J11500-s80 as the second homokaryon parent, or control strains, into compost that was spawned with commercial strain A-15. Within this experiment design, normal incompatibility interactions would not interfere with overall mushroom yield, whereas strains with J15987's antagonism trait would cause significantly lower, even zero, yields of A-15.

Sixteen F2 strains were chosen for this study (see Table III below), with J15987 and A15 used as positive and negative controls respectively. Yields of A-15 in replicated confrontation crop tests were obtained. In a normal crop of Agaricus bisporus, the first break of mushrooms is harvested over a period of three to four days. Skilled harvesters pick the earliest mushrooms and leave spaces for the mushrooms to fill in as the crop develops. In contrast, when the AI trait is expressed, very few mushrooms are produced. Large expanses of the mushroom beds are barren and there are also some areas where a small number of can mushrooms appear. In other words, the AI trait results in a catastrophic reduction in mushroom yield. Generally, heterokaryon incompatibility or vegetative incompatibility may have yield reductions of 10% or less, and often 5% or less in most instances. However, the AI trademark is so catastrophic that reductions or more than 50% are not uncommon. However, for purposes of this invention, the AI trait will be defined as having a yield reduction of 15% or less of the A15 control yield after testing.

The next step was to look for a correlation between Aggressive Incompatibility and the chromosomes present in Strain #1, Strain #16 and J15987. To determine the genotype of each strain's parental J19109 homokaryon one amplicon per chromosome was amplified using PCR. These markers were located as close to the center of each chromosome as possible. Note that chromosomes of A. bisporus are very commonly inherited as an unrecombined unit and can be tracked to a first approximation by a single centrally located marker. In Table III below, we characterized nine chromosomes according to which parent (B18287-s82 or WBL-s290) contributed each chromosome.

Agaricus bisporus has thirteen chromosomes per homokaryon genome (Foulogne-Oriol et al. 2010). Four chromosomes were not included in the study analysis:
1) Chromosome 1 contains the MAT locus, which controls mating in Agaricus bisporus. The cohort of J19109 test lines was pre-screened for the MAT-1 allele, because the other 50% of lines obtained were not compatible with J11500-s80.
2) Chromosome 3 was 100% skewed to the WBL-s290 allele.
3) Chromosome 6 was 100% skewed to the WBL-s290 allele.
4) Chromosome 12 was 100% skewed to the B18287-s82 allele.

Extreme skew, as with the latter three chromosomes, likely results from the effect of a deleterious allele, but in any case, there is effectively no observed segregation at that locus, so no analysis of correlation of genotype to phenotype is possible. But because both marker segregation and phenotype segregation were observed in the analysis carried out, it appears that the four chromosomes listed above play no primary role, or no role at all, in conferring the AI trait phenotype.

For this study, there were therefore an informative panel of nine chromosomes.

TABLE III
F2 Hybrid Strain Yields vs observed genotypes of J19109 derived SSIs compared to J15987; Trial 21-72

| | | A15 | Alleles: | | | | | | | | |
|---|---|---|---|---|---|---|---|---|---|---|---|
| Strain | Yield | ratio | C2 | C4 | C5 | C7 | C8 | C9 | C10 | C11 | C13 |
| J15987 | 0 | 0.00 | s290 | s290 | s290 | s290 | s290 | s290 | s290 | s290 | s290 |
| Strain #1 | 0 | 0.00 | s290 | s290 | s290 | s290 | s82 | s290 | s82 | s82 | s82 |
| Strain #2 | 7.18 | 0.97 | s82 | s82 | s290 | s82 | s290 | s82 | s290 | s82 | s290 |
| Strain #3 | 7.01 | 0.95 | s290 | s290 | s82 | s82 | s290 | s290 | s290 | s290 | s290 |
| Strain #4 | 7.47 | 1.01 | s290 | s82 | s82 | s82 | s290 | s290 | s290 | s290 | s290 |
| Strain #5 | 3.93 | 0.53 | s82 | s290 | s290 | s82 | s82 | s82 | s290 | s82 | s82 |
| J20176 | 7.74 | 1.05 | s290 | s82 | s82 | s290 | s82 | s290 | s290 | s290 | s82 |
| Strain #7 | 7.81 | 1.06 | s82 | s290 | s82 | s82 | s82 | s290 | s82 | s82 | s290 |
| Strain #8 | 7.28 | 0.99 | s82 | s82 | s290 | s82 | s290 | s82 | s82 | s290 | s82 |
| Strain #9 | 6.79 | 0.92 | s82 | s82 | s82 | s82 | s82 | s82 | s82 | s82 | s82 |
| Strain #10 | 7.23 | 0.98 | s290 | s82 | s290 | s290 | s82 | s290 | s82 | s82 | s290 |
| Strain #11 | 8.13 | 1.10 | s82 | s82 | s82 | s290 | s290 | s82 | s82 | s290 | s290 |
| Strain #12 | 7.22 | 0.98 | s82 | s290 | s290 | s82 | s290 | s290 | s290 | s82 | s290 |
| Strain #13 | 6.61 | 0.90 | s82 | s82 | s82 | s290 | s82 | s82 | s290 | s82 | s290 |
| Strain #14 | 7.45 | 1.01 | s290 | s290 | s290 | s82 | s290 | s290 | s82 | s290 | s290 |
| Strain #15 | 7.68 | 1.04 | s290 | s82 | s290 | s82 | s290 | s82 | s82 | s82 | s82 |
| Strain #16 | 1.08 | 0.15 | s82 | s290 | s82 | s290 | s82 | s290 | s82 | s82 | s82 |
| A15 | 7.38 | 1.00 | | | | | | | | | |

Data is in total pounds of mushrooms harvested in first break only
Mating#5 was affected by incidental disease which artificially lowered its yield
C refers to the chromosome number for each marker

Table III shows yields and inherited alleles for hybrid strains obtained from 16 different J19109 homokaryons individually mated with J11500-s80 to form F2 hybrid strains. This list includes J20176. In this test:

Three×2.76 sq. ft. containers (“tubs”) each were spawned with 55 lbs of Phase II mushroom compost for each strain. Yield was the total for three tubs.

In each treatment in the table, including J20176, 1% by weight of spawn of the relevant strain was thoroughly mixed with 99% A15 spawn, and the mixed spawn was thoroughly combined with the compost. Spawn run was complete at 15 days, and the casing layer was applied. For every treatment in Table III, the casing soil was inoculated with A15 was added (1.5% rate calculated using dry weights of casing soil).

Mushrooms appeared ready for harvest from day 16 to day 20, and the test concluded on day 20 with only the first break of production being harvested.

In column three the yield ratio is shown. This number was calculated by dividing total yield of each treatment by the A15 control yield.

Finally, the other columns in Table III display allelic inheritance of nine Agaricus chromosomes, complete with the allele inherited from either one or the other of the parents of J19109 (B18287-s82 and WBL-s290). Note that the parental homokaryons in the table are abbreviated to fit into the table: WBL-s290 is s290 and B18287-s82 is s82.

It can clearly be seen that in the presence of Strain #1, Strain #16 and J15987, the A15 crops had much lower yields than when traces of any of the other strains were present; in other words, these strains met the defined test criteria for manifestation of the AI trait in a test with A15. By utilizing the yield ratio data, it can de deduced that the Strain #1, Strain #16 and J15987 test treatments all had a yield ratio of 0.15 or lower. The closest strain to Strain #16 was Strain #5 which had a ratio of 0.53, however as noted above, this strain was badly affected by Trichoderma contamination, causing a yield reduction. Thus, a working definition for the AI trait in this test protocol was proposed to include a yield reduction to 15% or less of the A15 control yield.

Furthermore, when the patterns of chromosome inheritance were studied it became clear that Strain #1, Strain #16, and J15987 all shared the same WBL-s290 alleles at Chromosomes 4, 7, and 9. No other strain in this study shared this haplotype, strongly suggesting a correlation with the aggressive incompatibility trait.

The chances of a perfect agreement between chromosomes 4, 7 and 9 and the AI trait happening randomly can be calculated using Bernoulli's classic binomial probability formula,P=n×q^{x}(1−q)^{n-x} where P is the binomial probability, n is the total number of strains studied, x is the observed number of strains that match the specific genotype, and q is the probability of the specific genotype occurring per sample.

With 3 strains matching the specific genotype (two from the test cohort, plus J15987), 17 total strains studied, and a 0.125 probability of the genotype occurring for each strain (the observed segregations did not depart significantly from the theoretical 0.5 probability per allele; at three alleles, 0.5³=0.125), the calculated binomial probability for this scenario is 0.0154, or a 1.54% chance. In other words, the p-value for the possibility that the correlation between phenotype and haplotype was due to chance was 0.0154. Conventionally, a p-value of less than 0.05 is considered a strong basis for the rejection of a random chance effect.

However, 0.5 probability per allele as used above assumes a Mendelian 50:50 ratio.

The theoretical probability of 0.5 can be further refined using the data in Table III. For chromosome 4, there were 8/17 (=0.47:0.53) s290 alleles, for chromosome 7, 7/17 (=0.41:0.59) and for chromosome 9, 10 out of 17 (=0.41:0.59). For such a small sample size, the observed ratios agree well with the expectation of an ideal 50:50 ratio between each allele and its alternate. The total for all three chromosomes is thus 25/51, which is 0.49:0.51 (very close to a 50:50 ratio).

Additionally, because J15987 is part of a separate cohort, we could calculate the binomial probability without the J15987 data, in other words calculating for J20157 and J20209 only. This reduces the sample size to 16 from 17. The binomial probability in this case is 7.03%, which are still good odds (93%) that the chromosome association with the AI trait is real.

Taken together, the statistical analyses showed a high degree of probability that Aggressive Incompatibility is significantly correlated with, and can be explained by, the effects of incompatibility factors on three specific chromosomes from the WBL-s290 parent.

In conclusion, these data proved that the AI trait was under genetic control, and that it was possible to make new matings which retained a significant percentage of the genetic makeup (and phenotype) present in J15987, yet which were free of the AI trait. Finally, there is robust, statistically-supported segregation data to strongly suggest that AI is controlled by genetic factors on three different Agaricus chromosomes, allowing the claimed methods to be employed with high predictive confidence.

Table IV
Test 20-84; Yields of J19109 SSI × J11500-s80 matings

| Strain | 1st Break | 2nd Break | Total Yield | 1st: 2nd |
|---|---|---|---|---|
| Strain #3 | 3.5 | 2.02 | 5.52 | 1.73 |
| Strain #10 | 3.91 | 1.97 | 5.88 | 1.98 |
| Strain #14 | 3.72 | 2.37 | 6.09 | 1.57 |
| Strain #15 | 4.26 | 1.64 | 5.9 | 2.6 |
| Strain #17 | 3.64 | 1.72 | 5.36 | 2.12 |
| Strain #18 | 3.81 | 1.89 | 5.7 | 2.02 |
| A15 | 2.98 | 2.34 | 5.32 | 1.27 |
| J15987 | 4.2 | 1.79 | 5.99 | 2.35 |

Values in the table are lbs. per sq. ft.
Each value is the mean of six replicates per strain

TABLE V
Test 20-98; Yields of J19109 SSI × J11500-s80 matings

| Strain | 1st Break | 2nd Break | Total Yield | 1st: 2nd |
|---|---|---|---|---|
| Strain #4 | 3.8 | 1.6 | 5.4 | 2.38 |
| J20176 | 3.56 | 1.86 | 5.42 | 1.91 |
| Strain #7 | 3.17 | 1.4 | 4.57 | 2.26 |
| Strain #9 | 3.16 | 1.59 | 4.75 | 1.99 |
| Strain #12 | 3.99 | 1.59 | 5.58 | 2.51 |
| Strain #13 | 3.35 | 1.5 | 4.85 | 2.23 |
| A15 | 2.07 | 2.4 | 4.47 | 0.86 |
| J15987 | 3.54 | 1.7 | 5.25 | 2.08 |

Values in the table are lbs. per sq. ft.
Each value is the mean of six replicates per strain

Data in Tables IV and V above demonstrate the yield potential of twelve different J19109 SSI homokaryon×J11500-s80 hybrids grown in two separate tests. It is notable that this group of new hybrids all outyielded the A15 test control and were closer in performance to the J15987 control.

Additionally, the 1^{st}:2^{nd} break ratio was also calculated for both trials. It is clear that J15987 is heavily skewed toward first break. The J19109 hybrids also tend to be skewed to first break, to varying degrees.

One of the goals of this invention was to demonstrate that the high yields of J15987 were replicated in the J19109 derived crosses. This is important because yield potential in this range is required for commercial success of a strain. The data in Tables IV and V clearly show that this was achieved.

For the data in Table VI and Table VII, significance is indicated by asterisks. For p=0.05 or less, *; for p=0.01 or less, **; for p=0.001 or less, ***, of p=0.0001 or less, ****.

TABLE VI
Cap Shape Differences between J19109 crosses in test 21-30

| | Cap | p | Stem | p | Flesh | p |
|---|---|---|---|---|---|---|
| Strain | Roundness | value | Thickness | value | Thickness | value |
| J15987 | 0.7 | \ | 0.36 | \ | 0.35 | \ |
| Strain #1 | 0.65 | 1.79E−04*** | 0.34 | 0.08 | 0.37 | 1.18E−03** |
| Strain #2 | 0.66 | 0.01** | 0.35 | 0.37 | 0.39 | 1.33E−06**** |
| Strain #3 | 0.68 | 0.3 | 0.3 | 7.07E−07**** | 0.37 | 0.02* |
| Strain #4 | 0.62 | 5.05E−07**** | 0.36 | 0.92 | 0.33 | 0.05* |
| Strain #5 | 0.71 | 0.21 | 0.33 | 0.01 | 0.39 | 6.87E−05**** |
| J20176 | 0.68 | 0.18 | 0.35 | 0.44 | 0.34 | 0.17 |
| Strain #7 | 0.65 | 9.23E−04*** | 0.34 | 0.15 | 0.36 | 0.22 |
| Strain #8 | 0.69 | 0.59 | 0.33 | 1.69E−03** | 0.35 | 0.9 |
| Strain #9 | 0.62 | 9.39E−09**** | 0.34 | 0.06 | 0.35 | 0.8 |
| Strain #10 | 0.66 | 0.01** | 0.35 | 0.72 | 0.34 | 0.27 |
| Strain #11 | 0.67 | 0.03* | 0.33 | 0.22 | 0.34 | 0.66 |
| Strain #12 | 0.73 | 4.21E−03** | 0.32 | 1.80E−04*** | 0.41 | 4.78E−09**** |
| Strain #13 | 0.61 | 0.01** | 0.32 | 3.73E−05**** | 0.34 | 0.13 |
| Strain #14 | 0.7 | 0.73 | 0.31 | 2.49E−04*** | 0.33 | 0.01** |
| Strain #15 | 0.7 | 0.55 | 0.31 | 3.45E−06**** | 0.38 | 4.67E−04*** |
| Strain #16 | 0.64 | 1.49E−03** | 0.35 | 0.48 | 0.35 | 0.78 |
| A-15 | 0.65 | 3.06E−04*** | 0.38 | 0.04* | 0.36 | 0.14 |

Statistics were pair-wise t-test comparisons with the J15987 control. Statistical significance marked with *

It has further been demonstrated that the overall quality of mushroom shape and color in J15987 were replicated in the J19109 derived hybrid strains. The data in Table VI clearly shows that this was achieved.

Mushroom cap measurements were taken using Storm 3C301 digital calipers. Sample sizes of twenty medium sized mushrooms at commercial maturity (35-40 mm in diameter with closed veils) were harvested and measured to obtain values for cap diameter and cap height. The mushrooms were then cut in half vertically to measure flesh thickness and stem width. Ratios between these values were calculated to find Cap Roundness (cap height/cap diameter), Flesh Thickness (flesh thickness/cap height), and Stem Thickness (stem width/cap diameter).

Cap Roundness is an approximation of how spherical the mushroom cap appears. A larger value indicates a rounder mushroom, which is preferred by growers and customers on the basis of visual appeal. A smaller value indicates a flatter mushroom. All of the J19109 derived hybrid strains recorded in Table VI fall within a continuum around J15987, clearly demonstrating this trait has been retained.

Stem Thickness is the ratio of the mushroom's stem width to the diameter of its cap. A larger value indicates a wider stem. Different markets have their own preferences for stem thickness, with some preferring wider or narrower stems based on their needs. Nearly all of the J19109 derived hybrid strains recorded in Table VI showed slightly narrower stems than J15987, but this is not a universally positive or negative quality.

Flesh Thickness is an approximation of how much of the mushroom cap's volume is comprised of flesh tissue, as opposed to stem or gill tissue. A larger value indicates a higher ratio of flesh tissue, which is preferred by growers as a sign of quality. All of the J19109 derived hybrid strains recorded in Table VI fall within a continuum around J15987, clearly demonstrating this trait has been retained.

When these mushroom shape data are considered together, clearly J15987 shape was retained.

TABLE VII
L*a*b measurement in J19109 crosses (tests 20-43 & 20-49)

| Strain | L value | p value | a value | p value | b value | p value |
|---|---|---|---|---|---|---|
| J15987 | 93.96 | \ | 4.34 | \ | 4.76 | \ |
| Strain #1 | 96.84 | 8.81E−12**** | 4.65 | 2.37E−03** | 5.71 | 0.01** |
| Strain #2 | 93.47 | 0.1 | 4.48 | 0.2 | 5.56 | 0.03* |
| J20176 | 94.27 | 0.15 | 4.4 | 0.52 | 4.6 | 0.62 |
| Strain #17 | 97.22 | 2.56E−10**** | 4.51 | 0.17 | 5.62 | 0.03* |
| Strain #7 | 93.15 | 0.21 | 4.29 | 0.62 | 7.55 | 0.18 |
| Strain #19 | 96.8 | 2.75E−08**** | 4.47 | 0.33 | 5.88 | 0.02* |
| Strain #9 | 93.67 | 0.28 | 4.39 | 0.59 | 5.34 | 0.11 |
| Strain #10 | 94.1 | 0.54 | 4.43 | 0.33 | 4.65 | 0.74 |
| Strain #12 | 93.88 | 0.77 | 4.27 | 0.47 | 4.93 | 0.6 |
| Strain #13 | 93.18 | 0.16 | 4.33 | 0.95 | 5.52 | 0.06 |
| Strain #14 | 97.01 | 5.76E−12**** | 4.61 | 0.01** | 5.27 | 0.21 |
| Strain #15 | 97.11 | 3.54E−09**** | 4.39 | 0.67 | 5.86 | 0.01** |
| Strain #16 | 93.34 | 0.01** | 4.51 | 0.06 | 4.86 | 0.74 |
| A-15 | 94.89 | 0.04* | 4.88 | 3.66E−05**** | 7.09 | 1.65E−06**** |

Statistics were pair-wise t-test comparisons with the J15987 control.

Mushroom cap color was measured using a Minolta Chroma Meter CR-200. Sample size was twenty medium sized mushrooms of 30-40 mm in diameter. The L*a*b system was used, where “L” is a measure of brightness, with 100 being complete whiteness and 0 being complete blackness. For the other two measurements, “a” is a green/red axis, and “b” is a yellow/blue axis.

For “a”, red values align on the positive side of the common axis, and green values align on negative values. In a similar fashion, on the “b” axis, yellow values are positive and blue values are negative.

L value is an objective measure of how white a mushroom cap appears when it is undamaged. For white mushrooms, a higher value is more desirable. All of the J19109 derived hybrid strains recorded in Table VII fall within a continuum around J15987, clearly demonstrating this trait has been retained. In fact, several strains recorded L values significantly higher than J15987, suggesting an improvement to the trait rather than simply retaining it.

“a” value and “b” value readings contribute to the hue of the mushroom. None of the J19109 derived crosses recorded in Table VH differed significantly from J15987, indicating the color of the strain has been retained in these crosses.

TABLE VIII
Yields

| Strain | 1st Break | 2nd Break | 3rd Break | Total Yield |
|---|---|---|---|---|
| A15 | 3.56 | 2.62 | 1.28*** | 7.45 |
| J20176 | 3.78 | 2.5 | 0.96 | 7.24 |

Fourteen replicates per strain.
Yield is in lbs. per sq. ft.
Statistics was a pair-wise T-test

Table VIII shows the yields obtained from the first pre-commercial trial of J20176, complete with an A-15 control. Growing conditions were typical for European commercial growers using the “Dutch-style” system, with bulk Phase I and Phase II composting, and a bulk spawn run (Phase III). The room was flushed for the A-15 control, with industry standard conditions.

J20176 had a higher yield than the control in first break although this difference did not meet significance. The only observation that reached significance was third break, where A-15 had a higher third break yield than J20176. A total yield comparison did not show a significant difference between the two strains. These data demonstrate the yield potential of J20176 even under commercial growing conditions not optimized for the requirements of J20176 have essentially the same yields as strain A-15. Generally, to obtain good yield, a process must be refined over a long period of time. Observationally, the J20176 mushrooms were rounder and whiter than the A-15 control.

Special attention was paid to look for evidence of Aggressive Incompatibility in this test, given that J20176 and A-15 were present in adjacent areas of the same physical space. We observed a typical strain to strain interaction: in areas where colonized compost or casing had mixed together, a typical incompatibility reaction was seen, in which there was only a small area of no growth where the A-15 and J20176 strains grew into one another. The AI trait phenomenon was not provoked by J20176 under commercial conditions. These data are an indication of the market suitability and potential of J20176.

Although the invention has been described in terms of particular embodiments in this application, one of ordinary skill in the art, in light of the teachings herein, can generate additional embodiments and modifications without departing from the spirit of, or exceeding the scope of, the claimed invention. Accordingly, it is understood that the descriptions herein are proffered only to facilitate comprehension of the invention and should not be construed to limit the scope thereof.

What is claimed is:

1. A method for excluding an aggressive incompatibility (AI) trait from Agaricus bisporus mushroom strains, wherein it is known that mating a culture of a white mushroom line designated WBL-s290, a representative culture of the line having been deposited under NRRL Accession No. 68167 with a culture of a mushroom line designated J11500-s80, a culture of the line having been deposited under the NRRL Accession No. 68164, provides a hybrid mushroom strain designated J15987, a representative culture of the strain having been deposited under NRRL Accession No. 67646, that has the AI trait, the method comprising:
mating a culture of a hybrid mushroom line designated B18287-s82, a representative culture of the line having been deposited under NRRL Accession No. 68168, with a culture of the white mushroom line designated WBL-s290, to obtain an F1 hybrid mushroom strain designated J19109, a representative culture of the F1 strain having been deposited under NRRL Accession No. 68163;
fruiting a culture of the new F1 strain designated J19109 to obtain homokaryotic spores therefrom;
obtaining cultures of homokaryotic lines from the homokaryotic spores from F1 strain J19109 and selecting a culture of a homokaryotic line from the F1 strain J19109 and;
mating the culture of a homokaryotic line from F1 strain J19109 with a culture of the mushroom line designated J11500-s80, to obtain an F2 hybrid mushroom strain;
testing a culture of the F2 hybrid mushroom strain to determine the presence or absence of the AI trait, wherein, in the absence of the AI trait, the AI trait has been excluded from the F2 hybrid mushroom strain.

2. The method of claim 1, wherein the homokaryotic line from the F1 strain J19109 lacks the centromere-linked alleles of white mushroom line WBL-s290 on chromosomes 4, 7 and 9.

3. The method of claim 1, wherein the F2 hybrid strain excludes the AI trait, and provides at least two beneficial traits found in strain J15987 selected from the group consisting of a cap shape as round as the cap shape for strain J15987, stems as thick as the stems for strain J15987, flesh as thick as the flesh for strain J15987, and a redness (a) value on the L-a-b color measurement scale that is within 10% of the redness (a) value for the strain J15987.

4. The method of claim 1, wherein the culture of the homokaryotic line from F1 strain J19109 is a line culture designated J19109-s40, a representative culture of the line having been deposited under NRRL Accession No. 68165.

5. The method of claim 4, wherein the step of mating the culture of the homokaryotic line from F1 strain J19109 with a culture of the mushroom line designated J11500-s80, includes the step of mating the line culture designated J19109-s40 with the mushroom line designated J11500-s80, wherein a resultant F2 hybrid strain designated J20176, a representative culture of the strain having been deposited under NRRL Accession No. 68166, is produced.

6. The method of claim 5, wherein the F2 hybrid strain designated J20176 is free of the AI trait, and retains at least two beneficial traits found in strain J15987 selected from the group consisting of a cap shape as round as the cap shape for strain J15987, stems as thick as the stems for strain J15987, flesh as thick as the flesh for strain J15987, and a redness (a) value on the L-a-b color measurement scale that is within 10,% of the redness (a) value for the strain J15987.

7. A culture comprising at least one set of chromosomes of an Agaricus bisporus line B18278-s82, the culture of the line B18278-s82 having been deposited under the NRRL Accession Number 68168, wherein said chromosomes comprise all of the alleles of the line B18278-s82 at the sequence-characterized marker loci listed in Table I.

8. A culture derived from an initial culture, wherein said initial culture is a culture of claim 6, such that at least 75% of its genome or genotype is present in the genome or genotype of the initial culture of line B18287-s82, wherein a culture of the initial line has been deposited under NRRL Accession No. 68168, or wherein the culture is derived directly from the initial culture such that all of its genome or genotype is present in the genome or genotype of the initial culture of line B18287-s82.

9. A culture comprising at least one set of chromosomes of an Agaricus bisporus line WBL-s290, the culture of the line WBL-s290 having been deposited under the NRRL Accession Number 65167, wherein said chromosomes comprise all of the alleles of the line WBL-s290 at the sequence-characterized marker loci listed in Table I.

10. A culture derived from an initial culture, wherein said initial culture is a culture of claim 8, such that at least 75% of its genome or genotype is present in the genome or genotype of the initial culture of line WBL-s290, wherein a culture of the initial line has been deposited under NRRL Accession No. 68167, or wherein the culture is derived directly from the initial culture such that all of its genome or genotype is present in the genome or genotype of the initial culture of line WBL-s290.

11. A culture comprising at least one set of chromosomes of an Agaricus bisporus line J11500-s80, the culture of the line J11500-s80 having been deposited under the NRRL Accession Number 65164, wherein said chromosomes comprise all of the alleles of the line J11500-s80 at the sequence-characterized marker loci listed in Table I.

12. A culture derived from an initial culture, wherein said initial culture is a culture of claim 8, such that at least 75% of its genome or genotype is present in the genome or genotype of the initial culture of line J11500-s80, wherein a culture of the initial line has been deposited under NRRL Accession No. 68164, or wherein the culture is derived directly from the initial culture such that all of its genome or genotype is present in the genome or genotype of the initial culture of line J11500-s80.

13. A culture comprising at least one set of chromosomes of an Agaricus bisporus line J19109-s40, the culture of the line J19109-s40 having been deposited under the NRRL Accession Number 68165, wherein said chromosomes comprise all of the alleles of the line J19109-s40 at the sequence-characterized marker loci listed in Table I.

14. A culture derived from an initial culture, wherein said initial culture is a culture of claim 10, such that at least 75% of its genome or genotype is present in the genome or genotype of the initial culture of line J19109-s40, wherein a culture of the initial line has been deposited under NRRL Accession No. 68165, or wherein the culture is derived directly from the initial culture such that all of its genome or genotype is present in the genome or genotype of the initial culture of line J19109-s40.

15. A hybrid mushroom culture of Agaricus bisporus designated strain J19109, a representative culture of the strain having been deposited under NRRL Accession No. 68163.

16. A culture of Agaricus bisporus derived from an initial culture, wherein said initial culture is a culture of claim 12, such that at least 75% of its genome or genotype is present in the genome or genotype of the initial culture of strain J19109, wherein a culture of the strain has been deposited under NRRL Accession No. 68163, or wherein the culture is derived directly from the initial culture such that all of its genome or genotype is present in the genome or genotype of the initial culture of strain J19109.

17. A hybrid mushroom culture of Agaricus bisporus designated strain J20176, a representative culture of the strain having been deposited under NRRL Accession No. 68166.

18. A culture of Agaricus bisporus derived from an initial culture, wherein said initial culture is a culture of claim 15, such that at least 75% of its genome or genotype is present in the genome or genotype of the initial culture of strain J20176, wherein a culture of the strain has been deposited under NRRL Accession No. 68166, or wherein the culture is derived directly from the initial culture such that all of its genome or genotype is present in the genome or genotype of the initial culture of strain J20176.

19. Mushrooms obtained from the culture of claim 17.

20. A product incorporating the culture of claim 17, the product selected from the group consisting of mycelium, spawn, fresh or processed mushrooms, mushroom spores, mushroom spawn, mushroom preparations and extracts and fractions, mushroom pieces, mushroom inoculum, casing inoculum, casing spawn, casing soil, inoculated compost, colonized compost, post-cropped compost and friable particulate matter.

21. A part of the culture of claim 17, selected from the group consisting of hyphae, mushrooms, dormant spores, germinating spores, homokaryons, heterokaryons, cells, nuclei, and protoplasts.
